(19) United States
(12) Patent Application Publication
(10) Pub. No.: US 2010/0267774 A1
(43) Pub. Date: Oct. 21, 2010

(54) P38 MAP KINASE INHIBITORS

(75) Inventors: David Festus Charles Moffat, Oxfordshire (GB); Stéphane Pintat, Oxfordshire (GB); Stephen Davies, Oxfordshire (GB)

Correspondence Address: BANNER & WITCOFF, LTD., 1100 13th STREET, N.W., SUITE 1200, WASHINGTON, DC, 20005-4051, US

(73) Assignee: CHROMA THERAPEUTICS LTD., Abingdon, Oxfordshire (GB)

(21) Appl. No.: 12/741,535

(22) PCT Filed: Nov. 7, 2007
(86) PCT No.: PCT/GB07/04259

§ 371 Date: May 14, 2010

(51) Int. Cl. A61K 31/4412; C07D 213/72; C07D 409/04; A61K 31/4436; A61P 37/00; A61P 29/00; A61P 17/06; A61P 11/00

(52) U.S. Cl. 514/336; 546/297; 546/281.4; 514/349

(57) ABSTRACT

Compounds of formula (I) are inhibitors of p38 MAP kinase, and are therefore of utility in the treatment of, inter alia, inflammatory conditions including rheumatoid arthritis and COPD: formula (I) wherein: G is —N═ or —CH═; D is an optionally substituted divalent mono- or bi-cyclic aryl or heteroaryl radical having 5-13 ring members; R₆ is hydrogen or optionally substituted C₁-C₃ alkyl; P represents hydrogen and U represents a radical of formula (IA); or U represents hydrogen and P represents a radical of formula -A-(CH₂)_{z}—X¹-L¹-Y—NH—CHR₁R₂ wherein A represents an optionally substituted divalent mono- or bicyclic carbocyclic or heterocyclic radical having 5-13 ring members; z, Y, L¹, and X¹ are as defined in the specification; R₁ is a carboxylic acid group (—COOH), or an ester group which is hydrolysable by one or more intracellular esterase enzymes to a carboxylic acid group; and R₂ is the side chain of a natural or non-natural alpha amino acid.

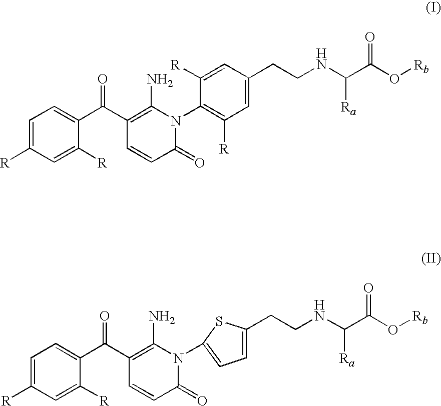

This invention relates to a series of amino acid and amino acid ester compounds, to compositions containing them, to processes for their preparation and to their use in medicine as p38 MAP kinase inhibitors for the treatment of autoimmune and inflammatory diseases, including rheumatoid arthritis, psoriasis, inflammatory bowel disease, Crohn's disease, ulcerative colitis, chronic obstructive pulmonary disease, asthma, multiple sclerosis, diabetes, atopic dermatitis, graft versus host disease, systemic lupus erythematosus and others.

### BACKGROUND OF THE INVENTION

Inappropriate activation of leukocytes including monocytes, macrophages and neutrophils leading to the production of elevated levels cytokines such as TNF-α, IL1-β and IL-8, is a feature of the pathogenesis of several inflammatory diseases including rheumatoid arthritis, ulcerative colitis, Crohn's disease, chronic obstructive pulmonary disease (COPD), asthma and psoriasis. The production of cytokines by inflammatory cells is a result of response to a variety of external stimuli, leading to the activation of a number of intracellular signalling mechanisms. Prominent amongst these is the mitogen-activated protein kinase (MAPK) superfamily consisting of highly conserved signalling kinases that regulate cell growth, differentiation and stress responses. Mammalian cells contain at least three families of MAPKs: the p42/44 extracellular signal-regulated kinase (ERK) MAPKs, c-Jun NH2-terminal kinases (JNKs) and p38 MAPK (also termed p38a/Mpk2/RK/SAPK2a/CSBP1/2). p38 MAPK was first cloned following its identification as a kinase that is tyrosine phosphorylated after stimulation of monocytes by lipopolysaccharide (LPS) [Han et al, Science 1994,265,808]. Additional homologues of mammalian p38 have been described and include p38β [Jiang et al, J. Biol. Chem., 1996, 271, 17920], p38γ [Li et al, Biochem. Biophys. Res. Commun., 1996, 228, 334] and p388δ [Jiang et al, J. Biol. Chem. 1997, 272, 30122]. While p38α and p38β are ubiquitously expressed, p38γ is restricted primarily to skeletal muscle and p38δ is predominantly expressed in lung and kidney.

The release of cytokines by host defence cells and the response of leukocytes to cytokines and other pro-inflammatory stresses are to varying extent regulated by p38 MAPK [Cuenda et al, FEBS Lett, 1995, 364, 229-233]. In other cell types, p38 MAPK controls stress responses such as the production of IL-8 by bronchial epithelial cells stimulated by TNF-α, and the up-regulation of the cell adhesion molecule ICAM-1 in LPS-stimulated endothelial cells. Upon activation, via dual phosphorylation of a TGY motif by the dual specificity kinases MKK3 and MKK6, p38 MAPK exerts its effects through phosphorylation of transcription factors and other kinases. MAP kinase-activated protein kinase-2 (MAPKAP-K2) has been identified as a target for p38 phosphorylation. It has been demonstrated that mice [Kotlyarov et al, Nat. Cell Biol. 1999, 1, 94-97] lacking MAPKAP-K2 release reduced levels of TNF-α, IL-1β, IL-6, IL-10 and IFN-γ in response to LPS/galactosamine mediated endotoxic shock. The regulation of the levels of these cytokines as well as COX-2 is at the mRNA level. TNF-α levels are regulated through translational control via AU-rich elements of the 3′-UTR of TNF-α mRNA, with MAPKAP-K2 signalling increasing TNF-α mRNA translation. MAPKAP-K2 signalling leads to increased mRNA stability for COX-2, IL-6 and macrophage inflammatory protein. MAPKAP-K2 determines the cellular location of p38 MAPK as well as transducing p38 MAPK signalling, possessing a nuclear localisation signal at its carboxyl terminus and a nuclear export signal as part of its autoinhibitory domain [Engel et al, EMBO J. 1998, 17, 3363-3371]. In stressed cells, MAPKAP-K2 and p38 MAPK migrate to the cytoplasm from the nucleus, this migration only occurring when p38 MAPK is catalytically active. It is believed that this event is driven by the exposure of the MAPKAP-K2 nuclear export signal, as a result of phosphorylation by p38 MAPK [Meng et al, J. Biol. Chem. 2002, 277, 37401-37405]. Additionally p38 MAPK either directly or indirectly leads to the phosphorylation of several transcription factors believed to mediate inflammation, including ATF1/2 (activating transcription factors 1/2), CHOP-10/GADD-153 (growth arrest and DNA damage inducible gene 153), SAP-1 (serum response factor accessory protein-1) and MEF2C (myocyte enhancer factor-2) [Foster et al, Drug News Perspect. 2000, 13, 488-497].

It has been demonstrated in several instances that the inhibition of p38 MAPK activity by small molecules, is useful for the treatment of several disease states mediated by inappropriate cytokine production including rheumatoid arthritis, COPD, asthma and cerebral ischemic. This modality has been the subject of several reviews [Salituro et al, Current Medicinal Chemistry, 1999, 6, 807-823 and Kumar et al, Nature Reviews Drug Discovery 2003, 2, 717-726].

Inhibitors of p38 MAPK have been shown to be efficacious in animal models of rheumatoid arthritis, such as collagen-induced arthritis in rat [Revesz et al, Biorg. Med. Chem. Lett., 2000, 10, 1261-1364] and adjuvant-induced arthritis in rat [Wadsworth et al, J. Pharmacol. Exp. Ther., 1999, 291, 1685-1691]. In murine models of pancreatitis-induced lung injury, pretreatment with a p38 MAPK inhibitor reduced TNF-α release in the airways and pulmonary edema [Denham et al, Crit. Care Med., 2000, 29, 628 and Yang et al, Surgery, 1999, 126, 216]. Inhibition of p38 MAPK before ovalbumin (OVA) challenge in OVA-sensitized mice decreased cytokine and inflammatory cell accumulation in the airways in an allergic airway model of inflammation, [Underwood et al, J. Pharmacol. Exp. Ther., 2000,293, 281]. Increased activity of p38 MAP kinase has been observed in patients suffering from inflammatory bowel disease [Waetzig et al, J. Immunol, 2002,168,5432-5351]. p38 MAPK inhibitors have been shown to be efficacious in rat models of cardiac hypertrophy [Behr et al, Circulation, 2001, 104, 1292-1298] and cerebral focal ischemia [Barone et al, J. Pharmacol. Exp. Ther., 2001, 296, 312-321].

In our co-pending International Patent Application No: PCT/GB2007/001596, we describe and claim compounds of formula (I):

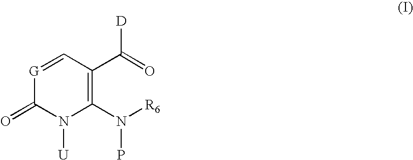

wherein:
G is —N═ or —CH═
D is an optionally substituted divalent mono- or bicyclic aryl or heteroaryl radical having 5-13 ring members;
R₆ is hydrogen or optionally substituted C₁-C₃ alkyl;
P represents hydrogen and U represents a radical of formula (IA); or U represents hydrogen and P represents a radical of formula (IA);

-A-(CH₂)_{z}—X¹-L¹-Y—NH—CHR₁R₂ (IA)

wherein
A represents an optionally substituted divalent mono- or bicyclic carbocyclic or heterocyclic radical having 5-13 ring members;
z is 0 or 1;
Y is a bond, —C(═O)—, —S(═O)₂—, —C(═O)NR₃—, —C(═S)—NR₃, —C(═NH)NR₃ or —S(═O)₂NR₃— wherein R₃ is hydrogen or optionally substituted C₁-C₆ alkyl;
L¹ is a divalent radical of formula -(Alk¹)ₘ(Q)ₙ(Alk²)ₚ- wherein m, n and p are independently 0 or 1,
   Q is (i) an optionally substituted divalent mono- or bicyclic carbocyclic or heterocyclic radical having 5-13 ring members, or (ii), in the case where both m and p are 0, a divalent radical of formula -X²-Q¹- or -Q¹-X²- wherein X² is —O—, S— or NR^{A}— wherein R^{A} is hydrogen or optionally substituted C₁-C₃ alkyl, and Q¹ is an optionally substituted divalent mono- or bicyclic carbocyclic or heterocyclic radical having 5-13 ring members,
   Alk¹ and Alk² independently represent optionally substituted divalent C₃-C₇ cycloalkyl radicals, or optionally substituted straight or branched, C₁-C₆ alkylene, C₂-C₆ alkenylene, or C₂-C₆ alkynylene radicals which may optionally contain or terminate in an ether (—O—), thioether (—S—) or amino (—NR^{A}—) link wherein R^{A} is hydrogen or optionally substituted C₁-C₃ alkyl; and
X¹ represents a bond; —C(═O); or —S(═O)₂—; —NR₄C(═O)—, —C(═O)NR₄—, —NR₄C(═O)NR₅—, —NR₄S(═O)₂—, or —S(═O)₂NR₄— wherein R₄ and R₅ are independently hydrogen or optionally substituted C₁-C₆ alkyl.
R₁ is a carboxylic acid group (—COOH), or an ester group which is hydrolysable by one or more intracellular esterase enzymes to a carboxylic acid group; and
R₂ is the side chain of a natural or non-natural alpha amino acid. Those compounds are stated to be potent and selective inhibitors of p38 MAPK (p38α, β, γ and δ) and the isoforms and splice variants thereof especially p38α, p38β and p38β2. The compounds are thus of use in medicine, for example in the treatment and prophylaxis of immune and inflammatory disorders described herein. The compounds are characterised by the presence in the molecule of the amino acid motif or amino acid ester motif —NH—CHR₁R₂ which is hydrolysable by an intracellular carboxylesterase. The compounds of the invention having the lipophilic amino acid ester motif cross the cell membrane, and are hydrolysed to the acid by the intracellular carboxylesterases. The polar hydrolysis product accumulates in the cell since it does not readily cross the cell membrane. Hence the p38 MAP kinase activity of the compound is prolonged and enhanced within the cell. The compounds of that invention are related to the p38 MAP kinase inhibitors encompassed by the disclosures in International Patent Application WO03076405 but differ there from in that they have the amino acid ester motif referred to above.

International Patent Application No: PCT/GB2007/001596 also disclosed that the compounds with which it is concerned include those which selectively accumulate in macrophages. Macrophages are known to play a key role in inflammatory disorders through the release of cytokines in particular TNFα and IL-1 (van Roon et al, Arthritis and Rheumatism, 2003, 1229-1238). In rheumatoid arthritis they are major contributors to the maintenance of joint inflammation and joint destruction. Macrophages are also involved in tumour growth and development (Naldini and Carraro, Curr Drug Targets Inflamm Allergy, 2005, 3-8). Hence agents that selectively target macrophage cell proliferation could be of value in the treatment of cancer and autoimmune disease. Targeting specific cell types would be expected to lead to reduced side-effects. The way in which the esterase motif is linked to the p38 kinase inhibitor determines whether it is hydrolysed, and hence whether or not it accumulates in different cell types. Specifically, macrophages contain the human carboxylesterase hCE-1 whereas other cell types do not. In the general formula (I) of PCT/GB2007/001596, when the nitrogen of the esterase motif R₁CH(R₂)NH— is not directly linked to a carbonyl (—C(═O)—), ie when Y is not a —C(═O), —C(═O)O— or —C(═O)NR₃— radical, the ester will only be hydrolysed by hCE-1 and hence the inhibitors will only accumulate in macrophages. Herein, unless “monocyte” or “monocytes” is specified, the term macrophage or macrophages will be used to denote macrophages (including tumour associated macrophages) and/or monocytes.

### BRIEF DESCRIPTION OF THE INVENTION

The present invention relates to a group of specific compounds falling within the general disclosures of PCT/GB2007/001596, but not specifically identified or exemplified therein. The present compounds have the utilities of the general class of PCT/GB2007/001596 compounds, and in particular display the macrophage selectivity property discussed above.

### DETAILED DESCRIPTION OF THE INVENTION

According to the invention there is provided a compound selected from the group consisting of:
*Cyclopentyl (2S)-[(2-{-4-[6-amino-5-(2,4-difluorobenzoyl)-2-oxopyridin-1(2H)-yl]phenyl}ethyl)amino](phenyl)acetate;
tert-butyl (2S)-{[2-(4-{6-amino-5-[(2,4-difluorophenyl)carbonyl]-2-oxopyridin-1(2H)-yl}phenyl)ethyl]amino}(phenyl)ethanoate;
Cyclopentyl (2S)-{[2-(4-{6-amino-5-[(2,4-difluorophenyl)carbonyl]-2-oxopyridin-1(2H)-yl}phenyl)ethyl]amino}(cyclohexyl)ethanoate;
tert-butyl (2S)-{[2-(4-{6-amino-5-[(2,4-difluorophenyl)carbonyl]-2-oxopyridin-1(2H)-yl}phenyl)ethyl]amino}(cyclohexyl)ethanoate;
Cyclopentyl N-[2-(4-{6-amino-5-[(2,4-difluorophenyl)carbonyl]-2-oxopyridin-1(2H)-yl}phenyl)ethyl]-L-valinate;
tert-butyl N-[2-(4-{6-amino-5-[(2,4-difluorophenyl)carbonyl]-2-oxopyridin-1(2H)-yl}phenyl)ethyl]-L-valinate;
Cyclopentyl N-[2-(4-{6-amino-5-[(2,4-difluorophenyl)carbonyl]-2-oxopyridin-1(2H)-yl}phenyl)ethyl]-3-methyl-L-valinate;
tert-butyl N-[2-(4-{6-amino-5-[(2,4-difluorophenyl)carbonyl]-2-oxopyridin-1(2H)-yl}phenyl)ethyl]-3-methyl-L-valinate;
Cyclopentyl N-[2-(4-{6-amino-5-[(2,4-difluorophenyl)carbonyl]-2-oxopyridin-1(2H)-yl}phenyl)ethyl]-D-leucinate;
tert-butyl N-[2-(4-{6-amino-5-[(2,4-difluorophenyl)carbonyl]-2-oxopyridin-1(2H)-yl}phenyl)ethyl]-D-leucinate;
Cyclopentyl N-[2-(4-{6-amino-5-[(2,4-difluorophenyl)carbonyl]-2-oxopyridin-1(2H)-yl}phenyl)ethyl]-O-tert-butyl-L-serinate;
tert-butyl N-[2-(4-{6-amino-5-[(2,4-difluorophenyl)carbonyl]-2-oxopyridin-1(2H)-yl}phenyl)ethyl]-O-tert-butyl-L-serinate;
(1R,2S,5S)-2-Isopropyl-5-methylcyclohexyl N-(2-{4-[6-amino-5-(2,4-difluorobenzoyl)-2-oxopyridin-1(2H)-yl]phenyl}ethyl)-L-leucinate;
(1S,2R,5S)-2-Isopropyl-5-methylcyclohexyl N-(2-{4-[6-amino-5-(2,4-difluorobenzoyl)-2-oxopyridin-1(2H)-yl]phenyl}ethyl)-L-leucinate;
Cyclopentyl N-[2-(4-{6-amino-5-[(2,4-difluorophenyl)carbonyl]-2-oxopyridin-1(2H)-yl}phenyl)ethyl]-O-tert-butyl-L-threoninate;
tert-butyl N-[2-(4-{6-amino-5-[(2,4-difluorophenyl)carbonyl]-2-oxopyridin-1(2H)-yl}phenyl)ethyl]-O-tert-butyl-L-threoninate;
*Cyclopentyl N-[2-(4-{6-amino-5-[(2,4-difluorophenyl)carbonyl]-2-oxopyridin-1(2H)-yl}phenyl)ethyl]-L-threoninate;
Cyclopentyl N-[2-(4-{6-amino-5-[(2,4-difluorophenyl)carbonyl]-2-oxopyridin-1(2H)-yl}phenyl)ethyl]-L-isoleucinate;
tert-butyl N-[2-(4-{6-amino-5-[(2,4-difluorophenyl)carbonyl]-2-oxopyridin-1(2H)-yl}phenyl)ethyl]-L-isoleucinate;
Cyclopentyl N-[2-(4-{6-amino-5-[(2,4-difluorophenyl)carbonyl]-2-oxopyridin-1(2H)-yl}phenyl)ethyl]-L-alaninate;
tert-butyl N-[2-(4-{6-amino-5-[(2,4-difluorophenyl)carbonyl]-2-oxopyridin-1(2H)-yl}phenyl)ethyl]-L-alaninate;
*Cyclopentyl N-[2-(4-{6-amino-5-[(2,4-difluorophenyl)carbonyl]-2-oxopyridin-1(2H)-yl}phenyl)ethyl]-L-phenylalaninate;
tert-butyl N-[2-(4-{6-amino-5-[(2,4-difluorophenyl)carbonyl]-2-oxopyridin-1(2H)-yl}phenyl)ethyl]-L-phenylalaninate;
*Cyclopentyl N-(2-{-4-[6-amino-5-(4-fluorobenzoyl)-2-oxopyridin-1(2H)-yl]-3,5-difluorophenyl}ethyl)-L-leucinate;
*tert-butyl N-(2-{-4-[6-amino-5-(4-fluorobenzoyl)-2-oxopyridin-1(2H)-yl]-3,5-difluorophenyl}ethyl)-L-leucinate;
Cyclopentyl (2S)-[(2-{-4-[6-amino-5-(4-fluorobenzoyl)-2-oxopyridin-1(2H)-yl]-3,5-difluorophenyl}ethyl)amino](phenyl)acetate;
tert-butyl (2S)-[(2-{4-[6-amino-5-(4-fluorobenzoyl)-2-oxopyridin-1(2H)-yl]-3,5-difluorophenyl}ethyl)amino](phenyl)acetate;
Cyclopentyl (2S)-[(2-{4-[6-amino-5-(4-fluorobenzoyl)-2-oxopyridin-1(2H)-yl]-3,5-difluorophenyl}ethyl)amino](cyclohexyl)acetate;
Cyclopentyl N-(2-{-4-[6-amino-5-(4-fluorobenzoyl)-2-oxopyridin-1(2H)-yl]-3,5-difluorophenyl}ethyl)-D-leucinate;
tert-butyl N-(2-{-4-[6-amino-5-(4-fluorobenzoyl)-2-oxopyridin-1(2H)-yl]-3,5-difluorophenyl}ethyl)-D-leucinate;
Cyclopentyl N-(2-{-4-[6-amino-5-(4-fluorobenzoyl)-2-oxopyridin-1(2H)-yl]-3,5-difluorophenyl}ethyl)-O-tert-butyl-L-serinate;
tert-butyl N-(2-{-4-[6-amino-5-(4-fluorobenzoyl)-2-oxopyridin-1(2H)-yl]-3,5-difluorophenyl}ethyl)-O-tert-butyl-L-serinate;
Cyclopentyl N-(2-{4-[6-amino-5-(2,4-difluorobenzoyl)-2-oxopyridin-1(2H)-yl]-3,5-difluorophenyl}ethyl)-L-leucinate;
*Cyclopentyl (2S)-[(2-{4-[6-amino-5-(2,4-difluorobenzoyl)-2-oxopyridin-1(2H)-yl]-3,5-difluorophenyl}ethyl)amino](phenyl)acetate;
*tert-butyl (2S)-[(2-{4-[6-amino-5-(2,4-difluorobenzoyl)-2-oxopyridin-1(2H)-yl]-3,5-difluorophenyl}ethyl)amino](phenyl)acetate;
Cyclopentyl (2S)-[(2-{4-[6-amino-5-(2,4-difluorobenzoyl)-2-oxopyridin-1(2H)-yl]-3,5-difluorophenyl}ethyl)amino](cyclohexyl)acetate;
tert-butyl N-(2-{4-[6-amino-5-(2,4-difluorobenzoyl)-2-oxopyridin-1(2H)-yl]-3,5-difluorophenyl}ethyl)-L-leucinate;
Cyclopentyl N-(2-{4-[6-amino-5-(2,4-difluorobenzoyl)-2-oxopyridin-1(2H)-yl]-3,5-difluorophenyl}ethyl)-D-leucinate;
tert-butyl N-(2-{-4-[6-amino-5-(2,4-difluorobenzoyl)-2-oxopyridin-1(2H)-yl]-3,5-difluorophenyl}ethyl)-D-leucinate;
Cyclopentyl N-(2-{4-[6-amino-5-(2,4-difluorobenzoyl)-2-oxopyridin-1(2H)-yl]-3,5-difluorophenyl}ethyl)-O-tert-butyl-L-serinate
tert-butyl N-(2-{4-[6-amino-5-(2,4-difluorobenzoyl)-2-oxopyridin-1(2H)-yl]-3,5-difluorophenyl}ethyl)-O-tert-butyl-L-serinate;
Cyclopentyl (2R)-[(2-{4-[6-amino-5-(2,4-difluorobenzoyl)-2-oxopyridin-1(2H)-yl]-3,5-difluorophenyl}ethyl)amino](phenyl)acetate;
Cyclopentyl N-[2-(4-{6-amino-5-[(2,4-difluorophenyl)carbonyl]-2-oxopyridin-1(2H)-yl}-3,5 difluorophenyl)ethyl]-L-valinate;
*Cyclopentyl (2S)-{[2-(4-{6-amino-5-[(2,4-difluorophenyl)carbonyl]-2-oxopyridin-1(2H)-yl}-3,5-difluorophenyl)ethyl]amino}(4-hydroxyphenyl)ethanoate;
*Cyclopentyl N-[2-(4-{6-amino-5-[(2,4-difluorophenyl)carbonyl]-2-oxopyridin-1(2H)-yl}-3,5-difluorophenyl)ethyl]-L-threoninate;
*Cyclopentyl (2S)-{[2-(4-{6-amino-5-[(2,4-difluorophenyl)carbonyl]-2-oxopyridin-1(2H)-yl}-3,5-difluorophenyl)ethyl]amino}(4-methoxyphenyl)ethanoate;
*Cyclopentyl (2S)-{[2-(4-{6-amino-5-[(2,4-difluorophenyl)carbonyl]-2-oxopyridin-1(2H)-yl}-3,5-difluorophenyl)ethyl]amino}(4-fluorophenyl)ethanoate;
*tert-butyl (2S)-{[2-(4-{6-amino-5-[(2,4-difluorophenyl)carbonyl]-2-oxopyridin-1(2H)-yl}-3,5-difluorophenyl)ethyl]amino}(4-fluorophenyl)ethanoate;
Cyclopentyl N-(2-{5-[6-amino-5-(2,4-difluorobenzoyl)-2-oxopyridin-1(2H)-yl]-2-thienyl}ethyl)-L-leucinate;
tert-butyl N-(2-{5-[6-amino-5-(2,4-difluorobenzoyl)-2-oxopyridin-1(2H)-yl]-2-thienyl}ethyl)-L-leucinate;
(2S)-[(2-{4-[6-amino-5-(2,4-difluorobenzoyl)-2-oxopyridin-1(2H)yl]-phenyl}ethyl)amino](phenyl)acetic acid;
(2S)-{[2-(4-{6-amino-5-[(2,4-difluorophenyl)carbonyl]-2-oxopyridin-1(2H)-yl}phenyl)ethyl]amino}(cyclohexyl)ethanoic acid;
N-[2-(4-{6-amino-5-[(2,4-difluorophenyl)carbonyl]-2-oxopyridin-1(2H)-yl}phenyl)ethyl]-L-valine;
N-[2-(4-{6-amino-5-[(2,4-difluorophenyl)carbonyl]-2-oxopyridin-1(2H)-yl}phenyl)ethyl]-3-methyl-L-valine;
N-[2-(4-{6-amino-5-[(2,4-difluorophenyl)carbonyl]-2-oxopyridin-1(2H)-yl}phenyl)ethyl]-D-leucine;
N-[2-(4-{6-amino-5-[(2,4-difluorophenyl)carbonyl]-2-oxopyridin-1(2H)-yl}phenyl)ethyl]-O-tert-butyl-L-serine;
N-[2-(4-{6-amino-5-[(2,4-difluorophenyl)carbonyl]-2-oxopyridin-1(2H)-yl}phenyl)ethyl]-L-serine;
N-[2-(4-{6-amino-5-[(2,4-difluorophenyl)carbonyl]-2-oxopyridin-1(2H)-yl}phenyl)ethyl]-L-threonine;
N-[2-(4-{6-amino-5-[(2,4-difluorophenyl)carbonyl]-2-oxopyridin-1(2H)-yl}phenyl)ethyl]-L-isoleucine;
N-[2-(4-{6-amino-5-[(2,4-difluorophenyl)carbonyl]-2-oxopyridin-1(2H)-yl}phenyl)ethyl]-L-alanine;
N-[2-(4-{6-amino-5-[(2,4-difluorophenyl)carbonyl]-2-oxopyridin-1(2H)-yl}phenyl)ethyl]-L-phenylalanine;
(2S)-[(2-{4-[6-amino-5-(4-fluorobenzoyl)-2-oxopyridin-1(2H)-yl]-3,5-difluorophenyl}ethyl)amino](phenyl)acetic acid;
N-(2-{4-[6-amino-5-(4-fluorobenzoyl)-2-oxopyridin-1(2H)-yl]-3,5-difluorophenyl}ethyl)-L-leucine;
N-(2-{4-[6-amino-5-(4-fluorobenzoyl)-2-oxopyridin-1(2H)-yl]-3,5-difluorophenyl}ethyl)-D-leucine;
N-(2-{4-[6-amino-5-(4-fluorobenzoyl)-2-oxopyridin-1(2H)-yl]-3,5-difluorophenyl}ethyl)-O-tert-butyl-L-serine;
(2S)-[(2-{4-[6-amino-5-(2,4-difluorobenzoyl)-2-oxopyridin-1(2H)-yl]-3,5-difluorophenyl}ethyl)amino](phenyl)acetic acid;
N-(2-{-4-[6-amino-5-(2,4-difluorobenzoyl)-2-oxopyridin-1(2H)-yl]-3,5-difluorophenyl}ethyl)-L-leucine;
N-(2-{4-[6-amino-5-(2,4-difluorobenzoyl)-2-oxopyridin-1(2H)-yl]-3,5-difluorophenyl}ethyl)-D-leucine;
N-(2-{-4-[6-amino-5-(2,4-difluorobenzoyl)-2-oxopyridin-1(2H)-yl]-3,5-difluorophenyl}ethyl)-O-tert-butyl-L-serine;
(2S)-{[2-(4-{6-amino-5-[(2,4-difluorophenyl)carbonyl]-2-oxopyridin-1(2H)-yl}-3,5-difluorophenyl)ethyl]amino}(4-fluorophenyl)ethanoic acid and
N-(2-{5-[6-amino-5-(2,4-difluorobenzoyl)-2-oxopyridin-1(2H)-yl]-2-thienyl}ethyl)-L-leucine.

Of the above compounds, those marked with an asterisk are currently especially preferred.

Compounds of the invention above may be prepared in the form of salts, especially pharmaceutically acceptable salts, N-oxides, hydrates, and solvates thereof. Any claim to a compound herein, or reference herein to “compounds of the invention”, “compounds with which the invention is concerned”, “compounds of formula (I)” and the like, includes salts, N-oxides, hydrates, and solvates of such compounds.

In another broad aspect the invention provides the use of a compound of the invention in the preparation of a composition for inhibiting the activity p38 MAP kinase enzyme.

The compounds with which the invention is concerned may be used for the inhibition of p38 MAP kinase enzyme activity in vitro or in vivo.

In one aspect of the invention, the compounds of the invention may be used in the preparation of a composition for the treatment of autoimmune or inflammatory disease, particularly those mentioned above in which p38 MAP kinase activity plays a role.

In another aspect, the invention provides a method for the treatment of the foregoing disease types, which comprises administering to a subject suffering such disease an effective amount of a compound of the invention.

As used herein the term “salt” includes base addition, acid addition and quaternary salts. Compounds of the invention which are acidic can form salts, including pharmaceutically acceptable salts, with bases such as alkali metal hydroxides, e.g. sodium and potassium hydroxides; alkaline earth metal hydroxides e.g. calcium, barium and magnesium hydroxides; with organic bases e.g. N-methyl-D-glucamine, choline tris(hydroxymethyl)amino-methane, L-arginine, L-lysine, N-ethyl piperidine, dibenzylamine and the like. Those compounds (I) which are basic can form salts, including pharmaceutically acceptable salts with inorganic acids, e.g. with hydrohalic acids such as hydrochloric or hydrobromic acids, sulphuric acid, nitric acid or phosphoric acid and the like, and with organic acids e.g. with acetic, tartaric, succinic, fumaric, maleic, malic, salicylic, citric, methanesulphonic, p-toluenesulphonic, benzoic, benzenesulphonic, glutamic, lactic, and mandelic acids and the like. For a review on suitable salts, see Handbook of Pharmaceutical Salts: Properties, Selection, and Use by Stahl and Wermuth (Wiley-VCH, Weinheim, Germany, 2002).

The term ‘solvate’ is used herein to describe a molecular complex comprising the compound of the invention and a stoichiometric amount of one or more pharmaceutically acceptable solvent molecules, for example, ethanol. The term ‘hydrate’ is employed when said solvent is water.

As mentioned above, the compounds with which the invention is concerned are inhibitors of p38 MAK kinase activity, and are therefore of use in the treatment of diseases such as psoriasis, inflammatory bowel disease, Crohn's disease, ulcerative colitis, chronic obstructive pulmonary disease, asthma, multiple sclerosis, diabetes, atopic dermatitis, graft versus host disease, or systemic lupus erythematosus and rheumatoid arthritis, in which p38 MAP kinase activity plays a part.

It will be understood that the specific dose level for any particular patient will depend upon a variety of factors including the activity of the specific compound employed, the age, body weight, general health, sex, diet, time of administration, route of administration, rate of excretion, drug combination and the severity of the particular disease undergoing treatment. Optimum dose levels and frequency of dosing will be determined by clinical trial.

The compounds with which the invention is concerned may be prepared for administration by any route consistent with their pharmacokinetic properties. The orally administrable compositions may be in the form of tablets, capsules, powders, granules, lozenges, liquid or gel preparations, such as oral, topical, or sterile parenteral solutions or suspensions. Tablets and capsules for oral administration may be in unit dose presentation form, and may contain conventional excipients such as binding agents, for example syrup, acacia, gelatin, sorbitol, tragacanth, or polyvinyl-pyrrolidone; fillers for example lactose, sugar, maize-starch, calcium phosphate, sorbitol or glycine; tabletting lubricant, for example magnesium stearate, talc, polyethylene glycol or silica; disintegrants for example potato starch, or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, for example sorbitol, syrup, methyl cellulose, glucose syrup, gelatin hydrogenated edible fats; emulsifying agents, for example lecithin, sorbitan monooleate, or acacia; non-aqueous vehicles (which may include edible oils), for example almond oil, fractionated coconut oil, oily esters such as glycerine, propylene glycol, or ethyl alcohol; preservatives, for example methyl or propyl p-hydroxybenzoate or sorbic acid, and if desired conventional flavouring or colouring agents.

For topical application to the skin, the drug may be made up into a cream, lotion or ointment. Cream or ointment formulations which may be used for the drug are conventional formulations well known in the art, for example as described in standard textbooks of pharmaceutics such as the British Pharmacopoeia.

For topical application by inhalation, the drug may be formulated for aerosol delivery for example, by pressure-driven jet atomizers or ultrasonic atomizers, or preferably by propellant-driven metered aerosols or propellant-free administration of micronized powders, for example, inhalation capsules or other “dry powder” delivery systems. Excipients, such as, for example, propellants (e.g. Frigen in the case of metered aerosols), surface-active substances, emulsifiers, stabilizers, preservatives, flavorings, and fillers (e.g. lactose in the case of powder inhalers) may be present in such inhaled formulations. For the purposes of inhalation, a large number of apparata are available with which aerosols of optimum particle size can be generated and administered, using an inhalation technique which is appropriate for the patient. In addition to the use of adaptors (spacers, expanders) and pear-shaped containers (e.g. Nebulator®, Volumatic®), and automatic devices emitting a puffer spray (Autohaler®), for metered aerosols, in particular in the case of powder inhalers, a number of technical solutions are available (e.g. Diskhaler®, Rotadisk®, Turbohaler® or the inhalers for example as described in European Patent Application EP 0 505 321).

For topical application to the eye, the drug may be made up into a solution or suspension in a suitable sterile aqueous or non aqueous vehicle. Additives, for instance buffers such as sodium metabisulphite or disodium edeate; preservatives including bactericidal and fungicidal agents such as phenyl mercuric acetate or nitrate, benzalkonium chloride or chlorhexidine, and thickening agents such as hypromellose may also be included.

The active ingredient may also be administered parenterally in a sterile medium. Depending on the vehicle and concentration used, the drug can either be suspended or dissolved in the vehicle. Advantageously, adjuvants such as a local anaesthetic, preservative and buffering agent can be dissolved in the vehicle.

The compounds of the invention may be prepared according to the following Examples. All temperatures are in ° C. The following abbreviations are used

MeOH=methanol
EtOH=ethanol
EtOAc=ethyl acetate
Boc=tert-butoxycarbonyl
CDI=1,1′-carbonyl diimidazole
DCM=dichloromethane
DCE=dichloroethane
DMF=dimethylformamide
DMSO=dimethyl sulfoxide
TFA=trifluoroacetic acid
THF=tetrahydrofuran
Na₂CO₃=sodium carbonate
HCl=hydrochloric acid
DIPEA=diisopropylethylamine
NaH=sodium hydride
NaOH=sodium hydroxide
NaHCO₃=sodium hydrogen carbonate
Pd/C=palladium on carbon
TME=tert-butyl methyl ether
N₂=nitrogen
Na₂SO₄=sodium sulfate
Et₃N=triethylamine
NH₃=ammonia
TMSCl=trimethylchlorosilane
TBME=tertiary butyl methyl ether
NH₄Cl=ammonium chloride
NMP=1-methyl-2-pyrrolidinone
LiAlH₄=lithium aluminium hydride
MgSO₄=magnesium sulfate
ⁿBuLi=n-butyllithium
STAB=sodium triacetoxyborohydride
CO₂=carbon dioxide
EDCl=N-(3-Dimethylaminopropyl)-N′-ethylcarbodiimide hydrochloride
Et₂O=diethyl ether
LiOH=lithium hydroxide
HOBt=1-hydroxybenzotriazole

### ELS=Evaporative Light Scattering

TLC=thin layer chromatography
ml=milliliter(s)
g=gram(s)
mg=milligram(s)
mol=moles
mmol=millimole(s)
LCMS=high performance liquid chromatography/mass spectrometry
NMR=nuclear magnetic resonance
RT=room temperature

Microwave irradiation was carried out using a CEM Discover focused microwave reactor. Solvents were removed using a GeneVac Series I without heating or a Genevac Series II with VacRamp at 30° C. or a Buchi rotary evaporator. Purification of compounds by flash chromatography column was performed using silica gel, particle size 40-63 μm (230-400 mesh) obtained from Silicycle. Purification of compounds by preparative HPLC was performed on Gilson systems using reverse phase ThermoHypersil-Keystone Hyperprep HS C18 columns (12 μm, 100×21.2 mm), gradient 20-100% B (A=water/0.1% TFA, B=acetonitrile/0.1% TFA) over 9.5 min, flow=30 ml/min, injection solvent 2:1 DMSO:acetonitrile (1.6 ml), UV detection at 215 nm.

¹H NMR spectra were recorded on a Bruker 400 MHz AV or a Bruker 300 MHz AV spectrometer in deuterated solvents. Chemical shifts (δ) are in parts per million. Thin-layer chromatography (TLC) analysis was performed with Kieselgel 60 F₂₅₄ (Merck) plates and visualized using UV light.

Analytical LCMS was performed on Agilent HP1100, Waters 600 or Waters 1525 LC systems using reverse phase Hypersil BDS C18 columns (5 μm, 2.1×50 mm), gradient 0-95% B (A=water/0.1% TFA, B=acetonitrile/0.1% TFA) over 2.10 min, flow=1.0 ml/min. UV spectra were recorded at 215 nm using a Gilson G1315A Diode Array Detector, G1214A single wavelength UV detector, Waters 2487 dual wavelength UV detector, Waters 2488 dual wavelength UV detector, or Waters 2996 diode array UV detector. Mass spectra were obtained over the range m/z 150 to 850 at a sampling rate of 2 scans per second or 1 scan per 1.2 seconds using Micromass LCT with Z-spray interface or Micromass LCT with Z-spray or MUX interface. Data were integrated and reported using OpenLynx and OpenLynx Browser software.

### Intermediates

### Intermediate 1

### 4-chlorophenyl 3-(2,4-difluorophenyl)-3-oxopropanimidothioate

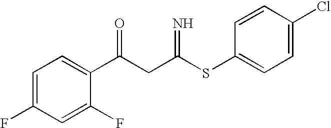

Intermediate 1 can be prepared using experimental procedures described in WO 2003076405.

### Intermediate 2

### {4-[6-amino-5-(2,4-difluorobenzoyl)-2-oxopyridin-1(2H)yl]-phenyl}acetaldehyde

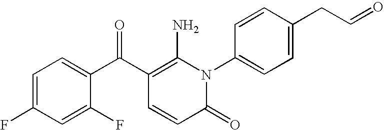

{4-[6-Amino-5-(2,4-difluorobenzoyl)-2-oxopyridin-1(2H)yl]-phenyl}acetaldehyde was synthesised using the route shown in Scheme 2 below.

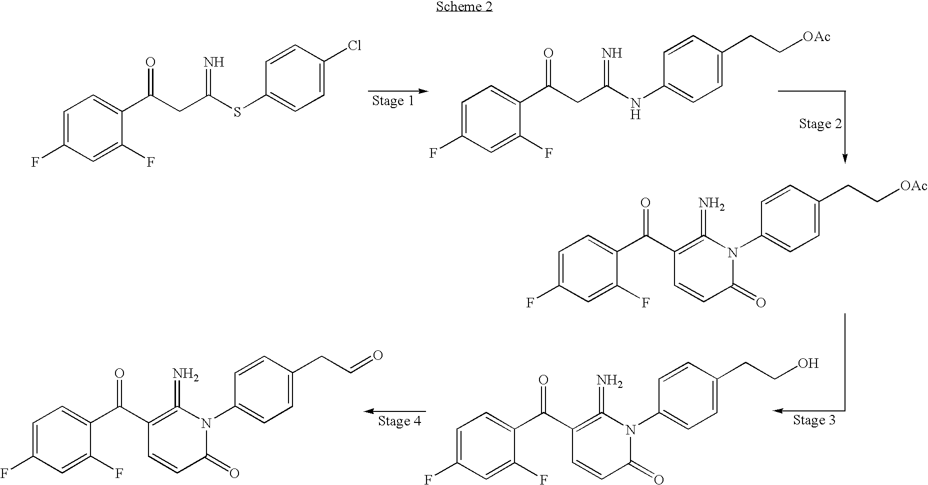

### Stage 1

### 2-(4-{[3-(2,4-difluorophenyl)-3-oxopropanimidoyl]amino}phenyl)ethyl acetate

4-chlorophenyl 3-(2,4-difluorophenyl)-3-oxopropanimidothioate (69.7 g, 192 mmol) was suspended in glacial acetic acid (700 ml) and 2-(4-aminophenyl)ethanol (27.71 g, 202 mmol, 1.05 eq) was added. The mixture was heated at 80° C. for 2.5 hrs before being allowed to cool to room temperature and concentrated under reduced pressure. The residue was triturated with Et₂O (500 ml) and washed with Et₂O (2×250 ml) to give a white solid, which was suspended in saturated NaHCO₃ (700 ml) and stirred vigorously for 30 minutes. Filtration and washing with water afforded a beige solid which was dried under reduced pressure to give the title compound (64.12 g, 92% yield).

LC/MS: m/z 361 [M+H]⁺. ¹H NMR (300 MHz, DMSO-d₆) δ: 7.79-7.71 (1H, m), 7.45-7.07 (6H, m), 5.26 (1H, s), 4.21 (2H, t, J=6.8 Hz), 2.89 (2H, t, J=6.5 Hz), 2.00 (3H, s).

### Stage 2

### 2-{4-[6-amino-5-(2,4-difluorobenzoyl)-2-oxopyridin-1(2H)-yl]phenyl}ethyl acetate

CDI (43.26 g, 267 mmol, 1.5 eq) was dissolved in anhydrous THF (1 l) under an atmosphere of nitrogen and cooled to 0° C. Propiolic acid (16.43 ml, 267 mmol, 1.5 eq) was added dropwise and the mixture allowed to warm to room temperature and stirred for 1 hr. A suspension of 2-(4-{[3-(2,4-difluorophenyl)-3-oxopropanimidoyl]-amino}phenyl)ethyl acetate (64.12 g, 178 mmol) in anhydrous THF (500 ml) was added and the mixture heated at 80° C. for 6 hrs before being left to stir at room temperature overnight. The resulting precipitate was collected by filtration, washed with Et₂O and dried under reduced pressure to give the title compound as a pale yellow solid (39.56 g). The filtrate was concentrated under reduced pressure to give a brown oil that was triturated with EtOAc (500 ml), providing a second batch of product by filtration (7.21 g). The two batches were combined to afford the title compound as a yellow solid (46.77 g, 64% yield).

LC/MS: m/z 413 [M+H]⁺. ¹H NMR (300 MHz, DMSO-d₆) δ: 7.55-7.37 (4H, m), 7.3-7.20 (4H, m), 5.72 (1H, d, J=9.6 Hz), 4.30 (2H, t, J=6.9 Hz), 3.01 (2H, t, J=6.9 Hz), 2.04 (3H, s).

### Stage 3

### 6-amino-5-(2,4-difluorobenzoyl)-1-[4-(2-hydroxyethyl)phenyl]pyridin-2(1H)-one

2-{-4-[6-amino-5-(2,4-difluorobenzoyl)-2-oxopyridin-1(2H)-yl]phenyl}ethyl acetate (46.77 g, 113 mmol) was suspended in 6N aqueous HCl (500 ml) and heated at reflux for 2 hrs. A precipitate formed upon cooling to room temperature which was collected by filtration, suspended in saturated aqueous NaHCO₃ (1000 ml) and stirred vigorously for 30 minutes. Filtration, washing with water (2×500 ml) and drying under reduced pressure afforded the title compound as an off-white solid (40.11 g, 96% yield).

LC/MS: m/z 371 [M+H]⁺. ¹H NMR (300 MHz, DMSO-d₆) δ: 7.55-7.37 (4H, m), 7.31-7.20 (4H, m), 5.71 (1H, d, J=9.9 Hz), 4.69 (1H, t, J=5.3 Hz), 3.71 (2H, m), 2.84 (2H, d, J=6.9 Hz).

### Stage 4

### {4-[6-amino-5-(2,4-difluorobenzoyl)-2-oxopyridin-1(2H)-yl]phenyl}-acetaldehyde

To a suspension of 6-amino-5-(2,4-difluorobenzoyl)-1-[4-(2-hydroxyethyl)phenyl]-pyridin-2(1H)-one (15.00 g, 40.5 mmol) in anhydrous DCM (750 ml) at 0° C. was added Dess-Martin Periodinane (20.62 g, 48.6 mmol, 1.2 eq) in portions. The mixture was allowed to warm to room temperature and stirred for 3 hrs, before being poured into saturated aqueous NaHCO₃ (400 ml) and saturated aqueous Na₂S₂O₃ (400 ml) and stirred vigorously for 30 minutes. The aqueous layer was separated and extracted with DCM (2×500 ml), and the organic extracts combined and dried over MgSO₄. Filtration and concentration under reduced pressure afforded the title compound as a crude pale yellow solid that was used without further purification (15.13 g).

LC/MS: m/z 369 [M+H]⁺.

### Intermediate 3

### 2-(4-Amino-3,5-difluoro-phenyl)-ethanol

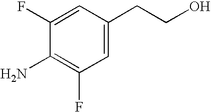

2-(4-Amino-3,5-difluoro-phenyl)-ethanol was synthesised using the route shown in Scheme 3 below.

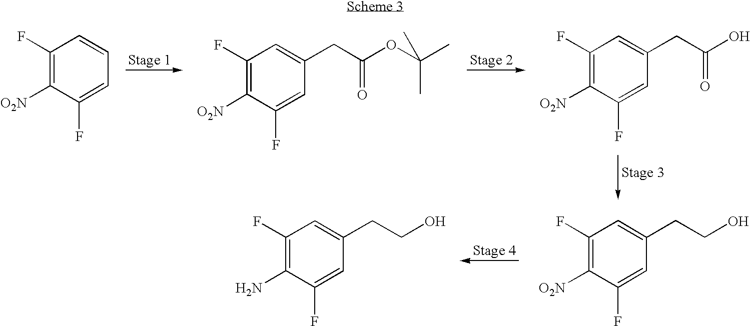

### Stage 1

### tert-butyl (3,5-difluoro-4-nitrophenyl)acetate

A mixture of potassium tert-butoxide (12.3 g, 111.0 mmol) in NMP (100 ml) was cooled to −20° C. under N₂. A mixture of 2,6-difluoronitrobenzene (5.0 g, 31.43 mmol) and tert-butylchloroacetate (7.6 ml, 53.11 mmol) in NMP (100 ml) was added slowly at −10° C. to −20° C. over 1.5 hrs. After 1.5 hrs the reaction was quenched by pouring into 2M HCl (120 ml) and ice, then heptane (300 ml) was added. The mixture was stirred for 10 minutes, separated and the aqueous extracted with heptane (2×400 ml). The organic layer was washed with brine twice, dried (MgSO₄), filtered and washed with heptane. The solution was concentrated in vacuo and the residue purified by column chromatography (3-4% EtOAc/Heptane) to provide the title compound as an orange oil (4.34 g, 53% yield).

¹H NMR (300 MHz, CDCl₃) δ: 7.06 (2H, d, J=8.7 Hz), 3.59 (2H, s), 1.48 (9H, s).

### Stage 2

### (3,5-difluoro-4-nitrophenyl)acetic acid

To a solution of tert-butyl (3,5-difluoro-4-nitrophenyl)acetate (4.34 g, 15.88 mmol) in DCM (10 ml), at 0° C., was added TFA (10 ml). The reaction was warmed to room temperature and stirred for 1.5 hrs. The reaction was concentrated in vacuo, slurried in heptane (10 ml), filtered and dried to provide the title compound as an orange solid (2.95 g, 86% yield).

¹H NMR (300 MHz, d₆DMSO) δ: 7.45 (2H, d, J=9.6 Hz), 3.79 (2H, s).

### Stage 3

### 2-(3,5-difluoro-4-nitrophenyl)ethanol

A solution of (3,5-difluoro-4-nitrophenyl)acetic acid (2.95 g, 13.59 mmol) in THF (30 ml), under N₂, was cooled to 0° C. and a solution of BH₃Me₂S in THF (10.2 ml, 20.38 mmol) was added dropwise over 5 minutes. The mixture was warmed to room temperature and stirred for 4.5 hrs. The reaction was cooled to 0° C. and quenched with MeOH (10 ml). The mixture was concentrated in vacuo and the residue purified by column chromatography (30-60% EtOAc/Hep) to provide the title compound as an oil (2.45 g, 89% yield).

¹H NMR (300 MHz, CDCl₃) δ: 7.03 (2H, d, J=9.3 Hz), 3.97-3.91 (2H, q, J=5.4, 5.7 Hz), 2.93 (2H, t, J=6.2 Hz), 1.52 (1H, t, J=5.0 Hz).

### Stage 4

### 2-(4-amino-3,5-difluorophenyl)ethanol

To a solution of 2-(3,5-difluoro-4-nitrophenyl)ethanol (2.45 g, 12.06 mmol) in EtOAc (50 ml) was added Pd/C (0.8 g). The mixture was stirred under an atmosphere of H₂ for 19 hrs, filtered and concentrated in vacuo to provide the title compound as a pale brown solid (2.15 g, 100% yield).

¹H NMR (300 MHz, CDCl₃) δ: 6.70-6.67 (2H, m), 3.82 (2H, t, J=6.5 Hz), 2.76 (2H, t, J=6.5 Hz).

### Intermediate 4

### 6-amino-1-[2,6-difluoro-4-(2-hydroxy-ethyl)-phenyl]-5-(4-fluoro-benzoyl)-1H-pyridin-2-one

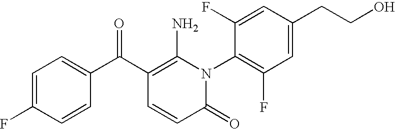

6-Amino-1-[2,6-difluoro-4-(2-hydroxy-ethyl)-phenyl]-5-(4-fluoro-benzoyl)-1H-pyridin-2-one was synthesised using the route shown in Scheme 4 below.

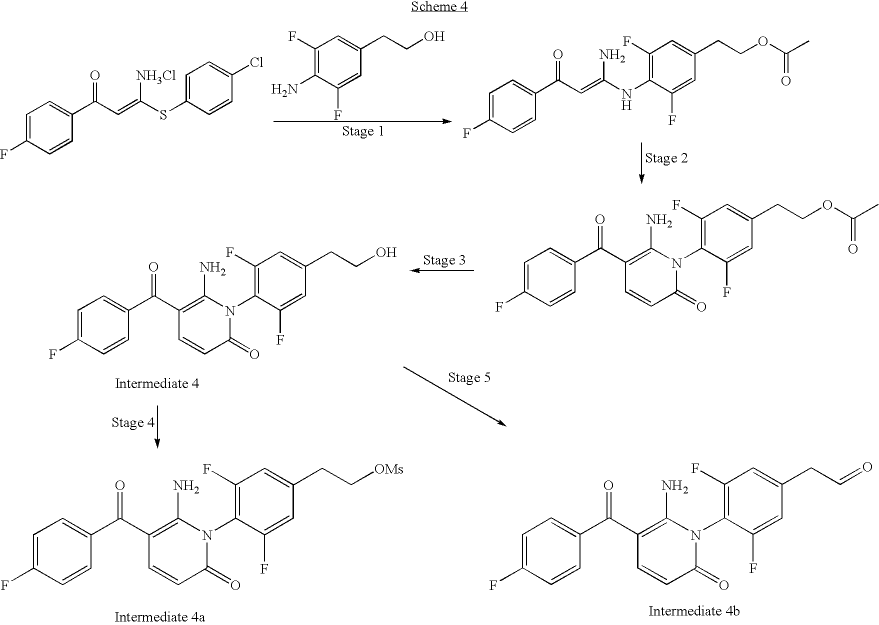

### Stage 1

### 2-(4-{[1-amino-3-(4-fluorophenyl)-3-oxoprop-1-en-1-yl]amino}-3,5-difluorophenyl)ethyl acetate

To a mixture of 3-amino-3-[(4-chlorophenyl)thio]-1-(4-fluorophenyl)prop-2-en-1-one hydrochloride (2.88 g, 8.36 mmol) in acetic acid (20 ml) was added 2-(4-amino-3,5-difluorophenyl)ethanol (1.52 g, 8.76 mmol) and the mixture heated at 80° C. for 20 hrs. The mixture was cooled, concentrated in vacuo and the residue triturated in diethyl ether to provide a solid. The solid was partitioned between EtOAc and sat NaHCO₃, washed with brine, dried (MgSO₄) and concentrated in vacuo to provide the title compound as a solid (2.49 g, 69% yield).

LC/MS: m/z 379.1 [M+H]⁺.

### Stage 2

### 2-{-4-[6-amino-5-(4-fluorobenzoyl)-2-oxopyridin-1(2H)-yl]-3,5-difluorophenyl}ethyl acetate

To a solution of CDI (1.61 g, 9.91 mmol) in THF (30 ml), under N₂ at 0° C., was added dropwise propiolic acid (611 μl, 9.91 mmol). The mixture was warmed to room temperature and stirred for 1.5 hrs. A solution of 2-(4-{[1-amino-3-(4-fluorophenyl)-3-oxoprop-1-en-1-yl]amino}-3,5-difluorophenyl)ethyl acetate (2.50 g, 6.62 mmol) in THF (15 ml) was added dropwise and the mixture heated at 80° C. for 5 hrs. The mixture was cooled, concentrated in vacuo and the residue purified by column chromatography (0.7-1% MeOH/DCM) to provide the title compound as a solid (1.30 g, 48% yield).

¹H NMR (300 MHz, CDCl₃) δ: 7.68-7.57 (3H, m), 7.22-7.15 (2H, m), 7.09 (2H, d, J=8.1 Hz), 5.92 (1H, d, J=9.9 Hz), 4.37 (2H, t, J=6.4 Hz), 3.06 (2H, t, J=6.4 Hz), 2.10 (3H, s).

### Stage 3

### 6-amino-5-(4-fluorobenzoyl)-1-[2,6-difluoro-4-(2-hydroxyethyl)phenyl]pyridin-2(1H)-one

To a mixture of 2-{4-[6-amino-5-(4-fluorobenzoyl)-2-oxopyridin-1(2H)-yl]-3,5-difluorophenyl}ethyl acetate (1.1 g, 2.45 mmol) in 6N aq HCl (50 ml) was heated at reflux for 24 hrs. The mixture was cooled, filtered and washed with water. The precipitate was partitioned between EtOAc and sat NaHCO₃, the organic layer washed with brine, dried (MgSO₄) and concentrated in vacuo to provide the title compound as a solid (790 mg, 80% yield).

LC/MS: m/z 389.1 [M+H]⁺.

### Stage 4

### 2-{-4-[6-amino-5-(4-fluorobenzoyl)-2-oxopyridin-1(2H)-yl]-3,5-difluorophenyl}ethyl methanesulfonate (Intermediate 4a)

To a solution of 6-amino-5-(4-fluorobenzoyl)-1-[2,6-difluoro-4-(2-hydroxyethyl)phenyl]pyridin-2(1H)-one (425 mg, 1.09 mmol) in DCM (10 ml), under N₂ at 0° C., was added methanesulfonyl chloride (93 μl, 1.2 mmol) and NEt₃ (303 μl, 2.18 mmol). The reaction was warmed to room temperature and stirred for 1 hr. The reaction was diluted with DCM, washed with 10% aq citric acid, sat NaHCO₃, brine, dried (MgSO₄) and concentrated in vacuo to provide the title compound as a solid (480 mg, 94% yield).

¹H NMR (300 MHz, CDCl₃) δ: 7.67-7.57 (3H, m), 7.22-7.08 (4H, m), 5.91 (1H, d, J=9.9 Hz), 4.53 (2H, t, J=6.2 Hz), 3.04 (3H, s).

### Stage 5

### {4-[6-amino-5-(4-fluorobenzoyl)-2-oxopyridin-1(2H)-yl]-3,5-difluorophenyl}acetaldehyde (Intermediate 4b)

To a mixture of 6-amino-5-(4-fluorobenzoyl)-1-[2,6-difluoro-4-(2-hydroxyethyl)phenyl]pyridin-2(1H)-one (440 mg, 1.08 mmol) in DCM (30 ml) was added Dess-Martin periodinane (690 mg, 1.63 mmol). The mixture was stirred for 3 hrs, sat aq Na₂S₂O₃ (30 ml) and sat NaHCO₃ (30 ml) was added and the mixture stirred vigorously for 30 minutes. The organic layer was separated and the aqueous extracted with DCM. The organic layer was washed with brine, dried (MgSO₄) and concentrated in vacuo to provide the title compound as a solid (430 mg, 78% yield).

¹H NMR (300 MHz, CDCl₃) δ: 9.88 (1H, s), 7.68-7.57 (3H, m), 7.23-7.07 (4H, m), 5.92 (1H, d, J=9.6 Hz), 3.88 (2H, s).

### Intermediate 5

### {4-[6-amino-5-(2,4-difluoro-benzoyl)-2-oxo-2H-pyridin-1-yl]-3,5-difluoro-phenyl}-acetaldehyde

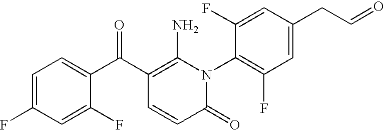

{4-[6-Amino-5-(2,4-difluoro-benzoyl)-2-oxo-2H-pyridin-1-yl]-3,5-difluoro-phenyl}-acetaldehyde was synthesised using the route shown in Scheme 5 below.

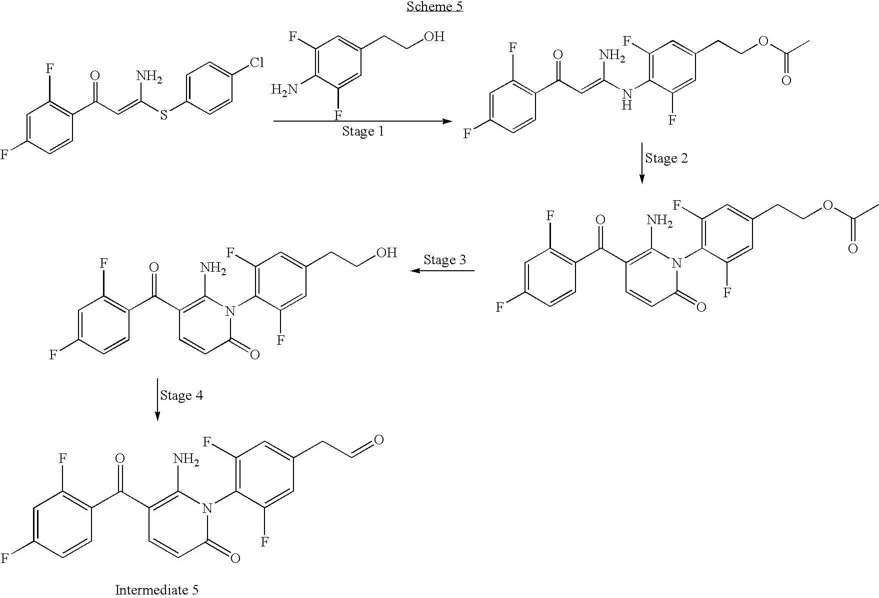

### Stage 1

### 2-(4-{[1-amino-3-(2,4-difluorophenyl)-3-oxoprop-1-en-1-yl]amino}-3,5-difluorophenyl)ethyl acetate

To a mixture of 3-amino-3-[(4-chlorophenyl)thio]-1-(2,4-difluorophenyl)prop-2-en-1-one hydrochloride (3.99 g, 11.1 mmol) in acetic acid (20 ml) was added 2-(4-amino-3, 5-difluorophenyl)ethanol (Intermediate 3) (2.00 g, 11.6 mmol) and the mixture heated at 80° C. for 20 hrs. The mixture was cooled, concentrated in vacuo and the residue triturated in diethyl ether to provide a solid. The solid was partitioned between EtOAc and sat NaHCO₃, washed with brine, dried (MgSO₄) and concentrated in vacuo to provide the title compound as a solid (2.91 g, 67% yield).

LC/MS: m/z 397 [M+H]⁺.

### Stage 2

### 2-{-4-[6-amino-5-(2,4-difluorobenzoyl)-2-oxopyridin-1(2H)-yl]-3,5-difluorophenyl}ethyl acetate

To a solution of CDI (1.78 g, 10.98 mmol) in THF (36 ml), under N₂ at 0° C., was added dropwise propiolic acid (675 μl, 10.98 mmol). The mixture was warmed to room temperature and stirred for 1.5 hrs. A solution of 2-(4-{[1-amino-3-(2,4-difluorophenyl)-3-oxoprop-1-en-1-yl]amino}-3,5-difluorophenyl)ethyl acetate (2.9 g, 7.32 mmol) in THF (18 ml) was added dropwise and the mixture heated at 80° C. for 5 hrs. The mixture was cooled, concentrated in vacuo and the residue purified twice by column chromatography (0.7-1% MeOH/DCM) to provide the title compound as a solid (1.20 g, 37% yield).

¹H NMR (300 MHz, CDCl₃) δ: 7.49-7.39 (2H, m), 7.09-6.90 (4H, m), 5.93 (1H, d, J=9.9 Hz), 4.37 (2H, t, J=6.4 Hz), 3.06 (2H, t, J=6.6 Hz), 2.10 (3H, s).

### Stage 3

### 6-amino-5-(2,4-difluorobenzoyl)-1-[2,6-difluoro-4-(2-hydroxyethyl)phenyl]pyridin-2(1H)-one

To a mixture of 2-{-4-[6-amino-5-(2,4-difluorobenzoyl)-2-oxopyridin-1(2H)-yl]-3,5-difluorophenyl}ethyl acetate (1.1 g, 2.45 mmol) in 6N aq HCl (50 ml) was heated at reflux for 24 hrs. The mixture was cooled, filtered and washed with water. The precipitate was partitioned between EtOAc and sat aq NaHCO₃, the organic layer further washed with brine, dried (MgSO₄) and concentrated in vacuo to provide the title compound as a solid (993 mg, 100% yield).

¹H NMR (300 MHz, CDCl₃) δ: 7.49-7.39 (2H, m), 7.15-6.90 (4H, m), 5.92 (1H, d, J=9.6 Hz), 4.00-3.85 (2H, m), 2.95 (2H, t, J=6.0 Hz).

### Stage 4

### {4-[6-amino-5-(2,4-difluorobenzoyl)-2-oxopyridin-1(2H)-yl]-3,5-difluorophenyl}acetaldehyde

To a mixture of 6-amino-5-(2,4-difluorobenzoyl)-1-[2,6-difluoro-4-(2-hydroxyethyl)phenyl]pyridin-2(1H)-one (500 mg, 1.23 mmol) in DCM (20 ml) was added Dess-Martin periodinane (783 mg, 1.85 mmol). The mixture was stirred for 3.5 hrs, sat aq Na₂S₂O₃ (20 ml) and sat NaHCO₃ (20 ml) was added and the mixture stirred vigorously for 30 minutes. The organic layer was separated and the aqueous extracted with DCM. The organic layer was washed with brine, dried (MgSO₄) and concentrated to provide the title compound as a solid (497 mg, 100% yield).

¹H NMR (300 MHz, CDCl₃) δ: 9.88 (1H, s), 7.49-7.40 (2H, m), 7.12-6.91 (4H, m), 5.93 (1H, d, J=9.9 Hz), 3.89 (2H, s).

### Intermediate 6

### 2-(5-amino-2-thienyl)ethyl acetate

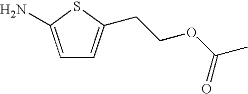

2-(5-Amino-2-thienyl)ethyl acetate was synthesised using the route shown in Scheme 6 below.

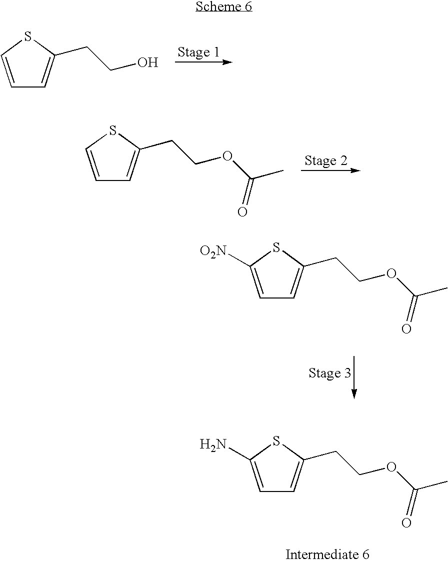

### Stage 1

### 2-(2-thienyl)ethyl acetate

To a solution of 2-thiophene ethanol (5 g, 39 mmol) in DCM (50 ml), at 0° C. under N₂, was added acetic anhydride (3.98 ml, 42.12 mmol), DIPEA (6.51 ml, 46.8 mmol) and DMAP (476 mg, 3.9 mmol). The reaction was warmed to room temperature and stirred for 3 hrs. The solution was washed with 5% HCl aq, 1M NaOH aq, brine, dried (MgSO₄) and concentrated in vacuo to provide the title compound as an oil (7.50 g, 100% yield).

¹H NMR (300 MHz, CDCl₃) δ: 7.20-7.17 (1H, m), 6.99-6.94 (1H, m), 6.89-6.87 (1H, m), 4.31 (2H, d, J=6.9 Hz), 3.18 (2H, d, J=6.8 Hz), 2.09 (3H, s).

### Stage 2

### 2-(5-nitro-2-thienyl)ethyl acetate

To a cold solution of acetic anhydride (2 ml), at −10° C., was added concentrated HNO₃ (118 μl) dropwise. The mixture was stirred for 20 minutes, then added to a cold solution of 2-(2-thienyl)ethyl acetate (300 mg, 1.76 mmol), acetic anhydride (3 ml), at −10° C., over 1 hr. The mixture was warmed to 0° C. and stirred for 1 hr, poured into ice water and extracted with DCM. The organic layer was washed with sat aq NaHCO₃, brine, dried (MgSO₄) and concentrated in vacuo. The residue was purified by column chromatography (7-20% EtOAc/Heptane) to provide the title compound as a solid (6.00 g, 68% yield).

¹H NMR (300 MHz, CDCl₃) δ: 7.81 (2H, d, J=4.2 Hz), 6.77 (2H, d, J=3.9 Hz), 4.34 (2H, d, J=6.3 Hz), 3.18 (2H, d, J=6.2 Hz), 2.11 (3H, s).

### Stage 3

### 2-(5-amino-2-thienyl)ethyl acetate

To a solution of 2-(5-nitro-2-thienyl)ethyl acetate (1 g, 4.65 mmol) in EtOAc (20 ml) was added Raney Ni (100 mg). The mixture was evacuated and stirred under an atmosphere of hydrogen for 18 hrs, filtered (Celite), washed with EtOAc and concentrated in vacuo. The residue was purified by column chromatography (20-30% EtOAc/Heptane) to provide the title compound as a solid (568 mg, 65% yield).

¹H NMR (300 MHz, CDCl₃) δ: 6.44 (2H, d, J=3.3 Hz), 6.06 (2H, d, J=3.6 Hz), 4.24 (2H, d, J=6.7 Hz), 2.99 (2H, d, J=6.6 Hz), 2.09 (3H, s).

### Intermediate 7

### {5-[6-amino-5-(2,4-difluorobenzoyl)-2-oxopyridin-1(2H)-yl]-2-thienyl}acetaldehyde

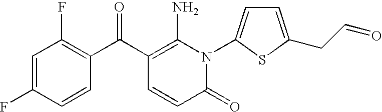

{5-[6-Amino-5-(2,4-difluorobenzoyl)-2-oxopyridin-1(2H)-yl]-2-thienyl}acetaldehyde was synthesised as shown below in Scheme 7.

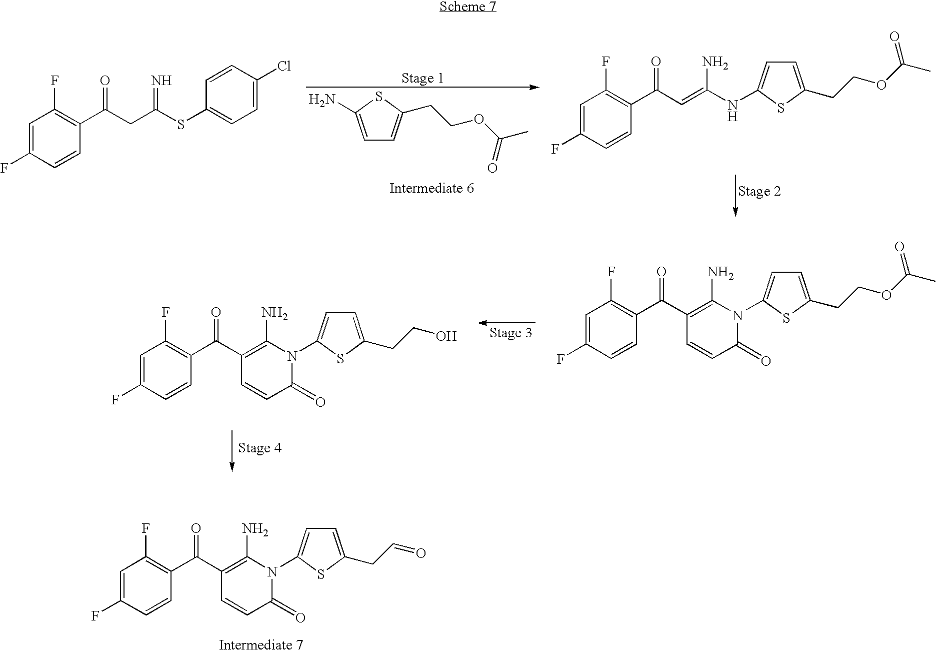

### Stage 1

### 2-(5-{[(1E/Z)-1-amino-3-(2,4-difluorophenyl)-3-oxoprop-1-en-1-yl]amino}-2-thienyl)ethyl acetate

To a solution of 3-amino-3-[(4-chlorophenyl)thio]-1-(2,4-difluorophenyl)prop-2-en-1-one hydrochloride (Intermediate 2) (1.82 g, 5.02 mmol) in acetic acid (15 ml), at 80° C., was added dropwise, over 1 hr, a solution of 2-(5-amino-2-thienyl)ethyl acetate (930 mg, 5.02 mmol) in acetic acid (10 ml). After 1 hr, a further 1 eq of 2-(5-amino-2-thienyl)ethyl acetate in acetic acid (10 ml) was added. The mixture was stirred for 3 hrs, cooled and concentrated in vacuo. The residue was partitioned between DCM/MeOH (4:1, 100 ml) and sat NaHCO₃. The organic layer was dried (MgSO₄), concentrated in vacuo and the residue purified by column chromatography (30-60% EtOAc/Heptane) to provide the title compound as a solid (547 mg, 34% yield).

LC/MS: m/z 367 [M+H]⁺.

### Stage 2

### 2-{5-[6-amino-5-(2,4-difluorobenzoyl)-2-oxopyridin-1(2H)-yl]-2-thienyl}ethyl acetate

As described for Stage 2 in Scheme 4.

¹H NMR (300 MHz, CDCl₃) δ: 10.61 (1H, br s), 7.46-7.33 (2H, m), 7.05-6.90 (4H, m), 5.90 (1H, d, J=9.6 Hz), 4.38 (2H, d, J=6.4 Hz), 2.21 (2H, d, J=6.3 Hz), 2.15 (3H, s).

### Stage 3

### 6-amino-5-(2,4-difluorobenzoyl)-1-[5-(2-hydroxyethyl)-2-thienyl]pyridin-2(1H)-one

As described for Stage 3 in Scheme 4.

LC/MS: m/z 377 [M+H]⁺.

### Stage 4

### {5-[6-amino-5-(2,4-difluorobenzoyl)-2-oxopyridin-1(2H)-yl]-2-thienyl}acetaldehyde

As described for Stage 5 in Scheme 4.

¹H NMR (300 MHz, CDCl₃) δ: 10.65 (1H, br s), 9.86 (1H, s), 7.47-7.30 (2H, m), 7.07-6.80 (4H, m), 5.91 (1H, d, J=9.6 Hz), 4.01 (2H, m).

### Intermediates 8 to 21

### Preparation of Aminoacid Esters

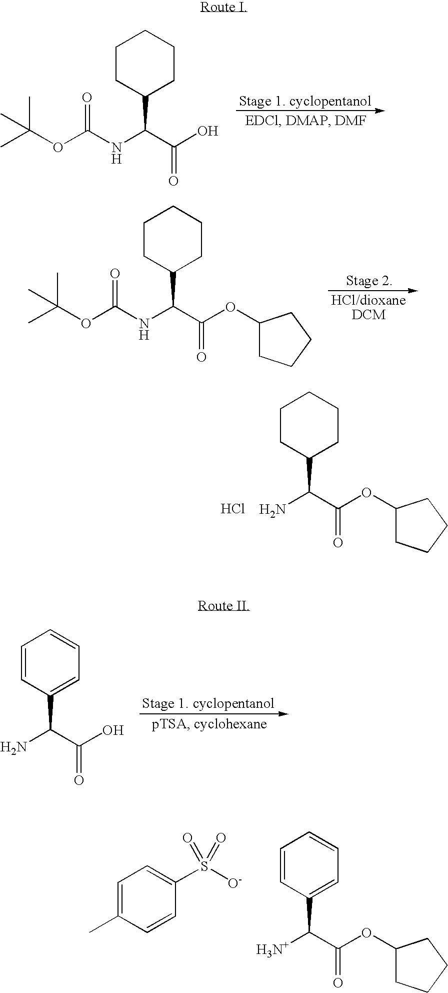

### Intermediates Prepared:

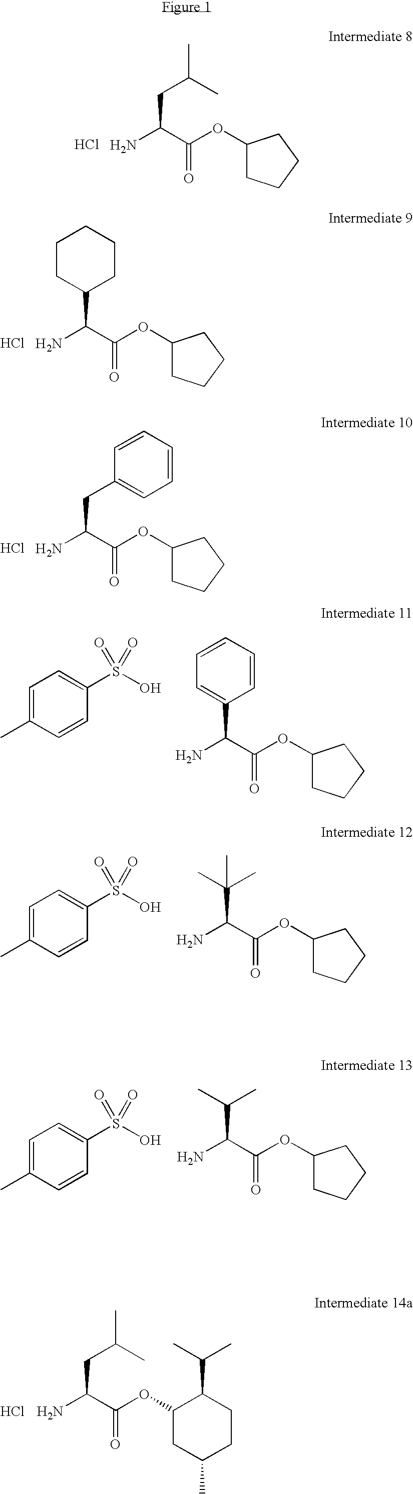

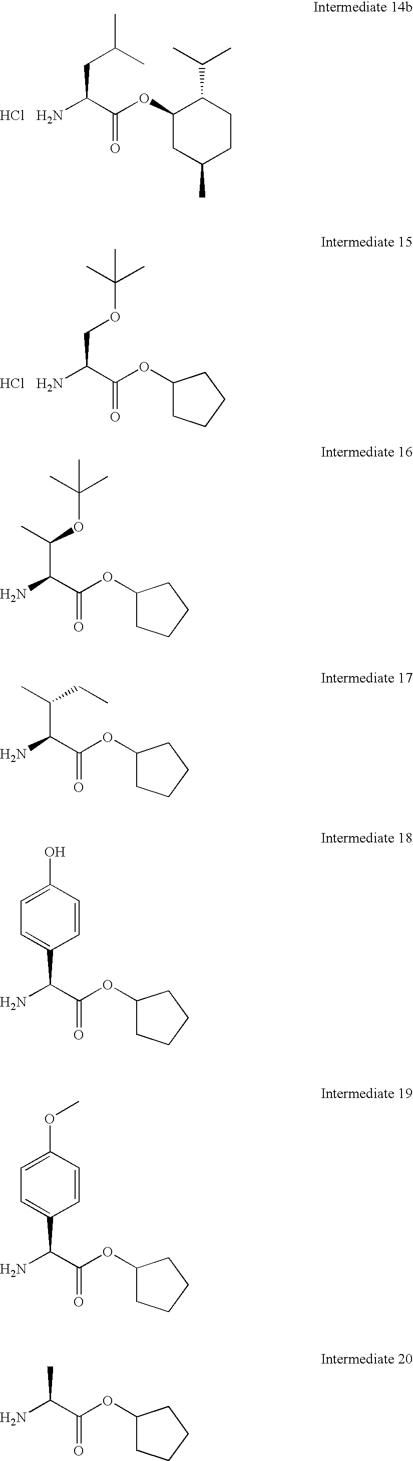

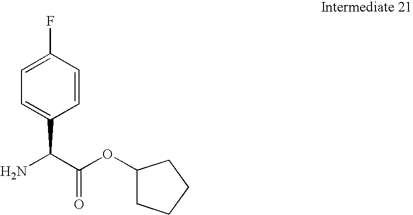

### Synthesis of Compounds Outlined in FIG. 1

### Route I (Exemplified for Intermediate 9)

### Stage 1-Ester Formation

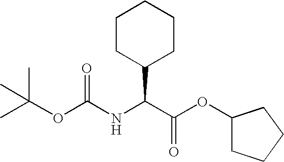

To a solution of (S)-2-tert-butoxycarbonylamino-3-cyclohexyl-propionic acid (5.00 g, 19.4 mmol) in DMF (50 ml) at 0° C. was added cyclopentanol (8.8 ml, 97.15 mmol), EDCI (4.09 g, 21.37 mmol) and finally DMAP (237 mg, 1.94 mmol). The reaction mixture was warmed to RT and stirred for 18 hr The DMF was removed in vacuo to give a clear oil. This was separated between water and EtOAc. The organic phase was dried (MgSO₄) and concentrated in vacuo. The crude extract was purified by column chromatography (25% EtOAc in heptane) to yield the desired product as a clear oil (14.87 g, 55% yield).

¹H NMR (300 MHz, DMSO-d₆) δ: 7.09 (1H, d), 5.08 (1H, t), 3.76 (1H, t), 1.50-1.85 (10H, br m), 1.39 (9H, s), 1.00-1.25 (9H, br m).

### Stage 2

### Cyclopentyl (2S)-amino(cyclohexyl)acetate hydrochloride (Intermediate 9)

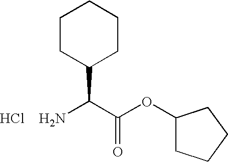

Stage 1 product (14.87 g, 45.69 mmol) was dissolved in DCM (100 ml) and treated with 4M HCl/dioxane (22.8 ml, 91.38 mmol) and the reaction mixture was stirred at RT for 24 hrs. The crude mixture was concentrated under reduced pressure to give an orange oil. This was triturated with Et₂O to give a white precipitate. This was further washed with Et₂O to give the desired product as a white powder (7.78 g, 65% yield).

¹H NMR (300 MHz, DMSO-d₆) δ: 8.45 (3H, br s), 5.22 (1H, t), 3.28 (1H, d), 1.95-1.50 (10H, br m), 1.30-0.90 (9H, br m).

### Route II (Exemplified for Intermediate 11)

### Stage 1

### (1S)-2-(cyclopentyloxy)-2-oxo-1-phenylethanaminium 4-methylbenzenesulfonate (Intermediate 11)

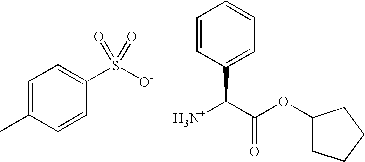

To a slurry of (S)-phenylglycine (5 g, 33.1 mmol) in cyclohexane (150 ml) was added cyclopentanol (29.84 ml, 331 mmol) and p-toluene sulfonic acid (6.92 g, 36.4 mmol). The reaction was fitted with a Dean-Stark receiver and heated to 135° C. for complete dissolution. After 12 hrs, the reaction was cooled to RT leading to the precipitation of a white solid. The solid was filtered and washed with EtOAc before drying under reduced pressure to give the required product as a white powder (11.01 g, 85% yield).

¹H NMR (300 MHz, DMSO-d₆) δ 8.82 (2H, br s), 8.73 (1H, br s), 7.47 (7H, m), 7.11 (2H, d), 5.25 (1H, br s), 5.18 (1H, m), 2.29 (3H, s), 1.87-1.36 (8H, m).

The corresponding (R)-amino acid esters of the above intermediates can be prepared in a similar manner to shown above, starting from the relevant commercially available (R)-amino acids. In addition, the corresponding tert-butyl esters are commercially available and are used directly where appropriate.

### EXAMPLES

### Example 1

### Cyclopentyl (2S)-[2-{4-[6-amino-5-(2,4-difluorobenzoyl)-2-oxopyridin-1(2H)-yl]phenyl}ethyl)amino](phenyl)acetate

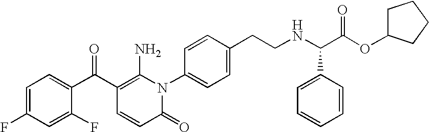

Example 1 was synthesised using Intermediate 2 and Intermediate 11 as described below.

To a solution of Intermediate 2 (130 mg, 0.35 mmol) in anhydrous THF (10 ml) were added cyclopentyl (2S)-amino(phenyl)acetate 4-methylbenzenesulfonate (Intermediate 11) (207 mg, 0.53 mmol, 1.5 eq) and NaBH(OAc)₃ (224 mg, 1.06 mmol, 3 eq). The mixture was stirred at room temperature for 16 hrs, and then quenched with water (20 ml). The aqueous layer was extracted with EtOAc (3×20 ml), and the combined organic extracts washed with brine (40 ml), dried over MgSO₄, filtered and concentrated under reduced pressure. The residue was triturated with Et₂O, collected by filtration and dried under reduced pressure to afford the title compound as a white solid (30 mg, 15% yield).

LC/MS: m/z 572 [M+H]⁺. ¹H NMR (300 MHz, DMSO-d₆) δ: 10.03 (2H, br s), 7.55-7.21 (13H, m), 5.71 (1H, d, J=9.6 Hz), 5.20 (2H, m), 3.08-2.94 (4H, m), 1.86-1.37 (8H, m).

The following examples were prepared in a similar manner to Example 1 using Intermediate 2 and the appropriate amino acid ester.

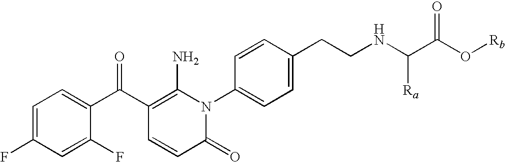

| Example | Intermediate | | | | |
|---|---|---|---|---|---|
| Number | used | Rₐ | R_{b} | Name | LCMS purity |
| 2 | 2 | 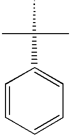 | tbutyl | tert-butyl (2S)-{[2-(4- {6-amino-5-[(2,4- difluorophenyl)carbon yl]-2-oxopyridin- 1(2H)- yl}phenyl)ethyl]amino} (phenyl)ethanoate | 91% purity: m/z 560 [M + H]⁺ |
| 3 | 2 + 9 | 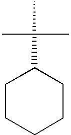 | cyclopentyl | Cyclopentyl (2S)-{[2- (4-{6-amino-5-[(2,4- difluorophenyl)carbon yl]-2-oxopyridin- 1(2H)- yl}phenyl)ethyl]amino} (cyclohexyl)ethanoate | 90% purity: m/z 578 [M + H]⁺ |
| 4 | 2 | 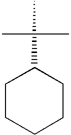 | tbutyl | tert-butyl (2S)-{[2-(4- {6-amino-5-[(2,4- difluorophenyl)carbon yl]-2-oxopyridin- 1(2H)- yl}phenyl)ethyl]amino} (cyclohexyl)ethanoate | 100% purity: m/z 566 [M + H]⁺ |
| 5 | 2 + 13 | 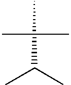 | cyclopentyl | Cyclopentyl N-[2-(4- {6-amino-5-[(2,4- difluorophenyl)carbon yl]-2-oxopyridin- 1(2H)- yl}phenyl)ethyl]-L- valinate | 95% purity: m/z 538 [M + H]⁺ |
| 6 | 2 | 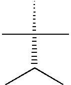 | tbutyl | tert-butyl N-[2-(4-{6- amino-5-[(2,4- difluorophenyl)carbon yl]-2-oxopyridin- 1(2H)- yl}phenyl)ethyl]-L- valinate | 100% purity: m/z 526 [M + H]⁺ |
| 7 | 2 + 12 | 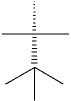 | cyclopentyl | Cyclopentyl N-[2-(4- {6-amino-5-[(2,4- difluorophenyl)carbon yl]-2-oxopyridin- 1(2H)- yl}phenyl)ethyl]-3- methyl-L-valinate | 95% purity: m/z 552 [M + H]⁺ |
| 8 | 2 | 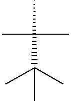 | tbutyl | tert-butyl N-[2-(4-{6- amino-5-[(2,4- difluorophenyl)carbon yl]-2-oxopyridin- 1(2H)- yl}phenyl)ethyl]-3- methyl-L-valinate | 95% purity: m/z 540 [M + H]⁺ |
| 9 | 2 | 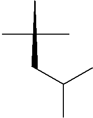 | cyclopentyl | Cyclopentyl N-[2-(4- {6-amino-5-[(2,4- difluorophenyl)carbon yl]-2-olxopyridin- 1(2H)- yl}phenyl)ethyl]-D- leucinate | 90% purity: m/z 552 [M + H]⁺ |
| 10 | 2 | 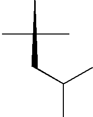 | tbutyl | tert-butyl N-[2-(4-{6- amino-5-[(2,4- difluorophenyl)carbon yl]-2-oxopyridin- 1(2H)- yl}phenyl)ethyl]-D- leucinate | 95% purity: m/z 540 [M + H]⁺ |
| 11 | 2 + 15 | 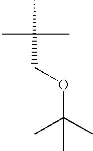 | cyclopentyl | Cyclopentyl N-[2-(4- {6-amino-5-[(2,4- difluorophenyl)carbon yl]-2-oxopyridin- 1(2H)- yl}phenyl)ethyl]-O- tert-butyl-L-serinate | 98% purity: m/z 582 [M + H]⁺ |
| 12 | 2 | 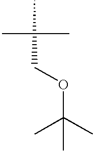 | tbutyl | tert-butyl N-[2-(4-{6- amino-5-[(2,4- difluorophenyl)carbon yl]-2-oxopyridin- 1(2H)- yl}phenyl)ethyl]-O- tert-butyl-L-serinate | 92% purity: m/z 570 [M + H]⁺ |
| 13 | 2 + 14a | 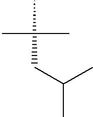 | 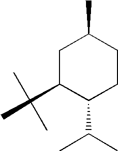 | (1R,2S,5S)-2- Isopropyl-5- methylcyclohexyl N- (2-{4-[6-amino-5-(2,4- difluorobenzoyl)-2- oxopyridin-1(2H)- yl]phenyl}ethyl)-L- leucinate | 97% purity: m/z 622 [M + H]⁺ |
| 14 | 2 + 14b | 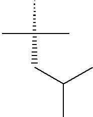 | 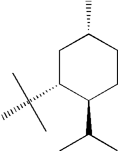 | (1S,2R,5S)-2- Isopropyl-5- methylcyclohexyl N- (2-{4-[6-amino-5-(2,4- difluorobenzoyl)-2- oxopyridin-1(2H)- yl]phenyl}ethyl)-L- leucinate | 95% purity: m/z 622 [M + H]⁺ |
| 15 | 2 + 16 | 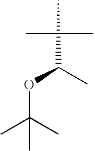 | cyclopentyl | Cyclopentyl N-[2-(4- {6-amino-5-[(2,4- difluorophenyl)carbon yl]-2-oxopyridin- 1(2H)- yl}phenyl)ethyl]-O- tert-butyl-L- threoninate | 95% purity: m/z 596 [M + H]⁺ |
| 16 | 2 | 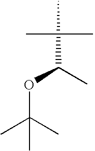 | tbutyl | tert-butyl N-[2-(4-{6- amino-5-[(2,4- difluorophenyl)carbon yl]-2-oxopyridin- 1(2H)- yl}phenyl)ethyl]-O- tert-butyl-L- threoninate | 98% purity: m/z 584 [M + H]⁺ |
| 17 | 2 | 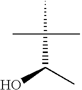 | cyclopentyl | Cyclopentyl N-[2-(4- {6-amino-5-[(2,4- difluorophenyl)carbon yl]-2-oxopyridin- 1(2H)- yl}phenyl)ethyl]-L- threoninate | 95% purity: m/z 540 [M + H]⁺ |
| 18 | 2 + 17 | 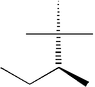 | cyclopentyl | Cyclopentyl N-[2-(4- {6-amino-5-[(2,4- difluorophenyl)carbon yl]-2-oxopyridin- 1(2H)- yl}phenyl)ethyl]-L- isoleucinate | 95% purity: m/z 552 [M + H]⁺ |
| 19 | 2 | 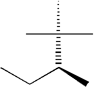 | tbutyl | tert-butyl N-[2-(4-{6- amino-5-[(2,4- difluorophenyl)carbon yl]-2-oxopyridin- 1(2H)- yl}phenyl)ethyl]-L- isoleucinate | 97% purity: m/z 540 [M + H]⁺ |
| 20 | 2 + 20 | 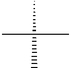 | cyclopentyl | Cyclopentyl N-[2-(4- {6-amino-5-[(2,4- difluorophenyl)carbon yl]-2-oxopyridin- 1(2H)- yl}phenyl)ethyl]-L- alaninate | 90% purity: m/z 510 [M + H]⁺ |
| 21 | 2 | 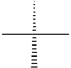 | tbutyl | tert-butyl N-[2-(4-{6- amino-5-[(2,4- difluorophenyl)carbon yl]-2-oxopyridin- 1(2H)- yl}phenyl)ethyl]-L- alaninate | 96% purity: m/z 498 [M + H]⁺ |
| 22 | 2 + 10 | 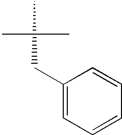 | cyclopentyl | Cyclopentyl N-[2-(4- {6-amino-5-[(2,4- difluorophenyl)carbon yl]-2-oxopyridin- 1(2H)- yl}phenyl)ethyl]-L- phenylalaninate | 99% purity: m/z 586 [M + H]⁺ |
| 23 | 2 | 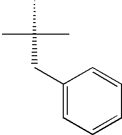 | tbutyl | tert-butyl N-[2-(4-{6- amino-5-[(2,4- difluorophenyl)carbon yl]-2-oxopyridin- 1(2H)- yl}phenyl)ethyl]-L- phenylalaninate | 97% purity: m/z 574 [M + H]⁺ |

### Example 24

### Cyclopentyl N-(2-{4-[6-amino-5-(4-fluorobenzoyl)-2-oxopyridin-1(2H)-yl]-3,5-difluorophenyl}ethyl)-L-leucinate

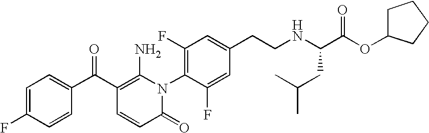

Example 24 was synthesised from Intermediate 4a as shown below.

To a mixture of cyclopentyl L-leucinate (Intermediate 8) (129 mg, 0.64 mmol), K₂CO₃ (89 mg, 0.643 mmol) and NaI (128 mg, 0.86 mmol) in DMF (1.5 ml) and THF (1.5 ml) was added 2-{-4-[6-amino-5-(4-fluorobenzoyl)-2-oxopyridin-1(2H)-yl]-3,5-difluorophenyl}ethyl methanesulfonate (Intermediate 4a) (200 mg, 0.43 mmol). The reaction was heated at 70° C. for 24 h, cooled and diluted with EtOAc. The organic layer was washed with sat NaHCO₃, brine, dried (MgSO₄) and concentrated in vacuo. The residue was purified by preparative HPLC to provide the title compound (45 mg, 11% yield).

LC/MS: m/z 570 [M+H]⁺. ¹H NMR (300 MHz, CD₃OD) δ: 7.57-7.48 (2H, m), 7.29 (2H, d, J=8.7 Hz), 7.20-7.05 (2H, m), 5.84 (1H, d, J=9.9 Hz), 4.02-3.96 (1H, m), 3.46-3.35 (2H, m), 3.21-3.14 (2H, m), 1.90-1.65 (3H, m), 1.57 (9H, s), 1.06 (6H, t, J=5.8 Hz).

### Example 25

### tert-butyl N-(2-{4-[6-amino-5-(4-fluorobenzoyl)-2-oxopyridin-1(2H)-yl]-3,5-difluorophenyl}ethyl)-L-leucinate

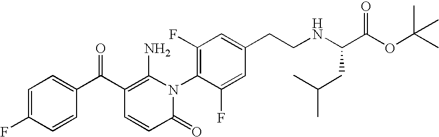

Example 25 was synthesised from Intermediate 4b as shown below.

To a solution of {4-[6-amino-5-(4-fluorobenzoyl)-2-oxopyridin-1(2H)-yl]-3,5-difluorophenyl}acetaldehyde (Intermediate 4b) (60 mg, 0.155 mmol) in DCE (2 ml) was added t-butyl L-leucinate (36 mg, 0.171 mmol), stirred for 30 minutes, and then STAB (80 mg, 0.377 mmol) was added. The reaction was stirred for 72 h, diluted with DCM and the organic layer washed with sat NaHCO₃, brine, dried (MgSO₄) and concentrated in vacuo. The residue was purified by column chromatography (0.75-1.25% MeOH/DCM), and then purified by preparative HPLC to provide the title compound (38 mg, 31% yield).

LC/MS: m/z 558 [M+H]⁺. ¹H NMR (300 MHz, CD₃OD) δ: 7.75 (1H, d, J=9.6 Hz), 7.67-7.60 (2H, m), 7.33-7.20 (4H, m), 5.84 (1H, d, J=9.9 Hz), 4.04-3.97 (1H, m), 3.48-3.30 (2H, m), 3.21-3.14 (2H, m), 1.90-1.70 (3H, m), 1.57 (9H, s), 1.06 (6H, t, J=6.0 Hz).

The following examples were synthesised as described above for Example 25, using Intermediate 4b and the appropriate amino acid ester.

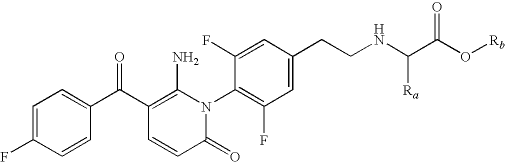

| Example | Intermediate | | | | |
|---|---|---|---|---|---|
| Number | used | Rₐ | R_{b} | Name | LCMS purity |
| 26 | 4b + 11 | 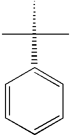 | cyclopentyl | Cyclopentyl (2S)-[(2-{4- [6-amino-5-(4- fluorobenzoyl)-2- oxopyridin-1(2H)-yl]- 3,5- difluorophenyl)ethyl)ami- no](phenyl)acetate | 95% purity: m/z 590 [M + H]⁺ |
| 27 | 4b | 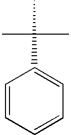 | tbutyl | tert-butyl (2S)-[(2-{4-[6- amino-5-(4- fluorobenzoyl)-2- oxopyridin-1(2H)-yl]- 3,5- difluorophenyl}ethyl)ami- no](phenyl)acetate | 95% purity: m/z 578 [M + H]⁺ |
| 28 | 4b + 9 | 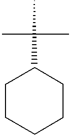 | cyclopentyl | Cyclopentyl (2S)-[(2-{4- [6-amino-5-(4- fluorobenzoyl)-2- oxopyridin-1(2H)-yl]- 3,5- difluorophenyl}ethyl)ami- no](cyclohexyl)acetate | 97% purity: m/z 596 [M + H]⁺ |
| 29 | 4b | 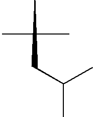 | cyclopentyl | Cyclopentyl N-(2-{4-[6- amino-5-(4- fluorobenzoyl)-2- oxopyridin-1(2H)-yl]- 3,5- difluorophenyl}ethyl)-D- leucinate | 99% purity: m/z 570 [M + H]⁺ |
| 30 | 4b | 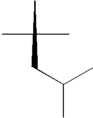 | tbutyl | tert-butyl N-(2-{4-[6- amino-5-(4- fluorobenzoyl)-2- oxopyridin-1(2H)-yl]- 3,5- difluorophenyl}ethyl)-D- leucinate | 99% purity: m/z 558 [M + H]⁺ |
| 31 | 4b + 15 | 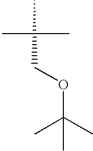 | cyclopentyl | Cyclopentyl N-(2-{4-[6- amino-5-(4- fluorobenzoyl)-2- oxopyridin-1(2H)-yl]- 3,5- difluorophenyl}ethyl)-O- tert-butyl-L-serinate | 96% purity: m/z 600 [M + H]⁺ |
| 32 | 4b | 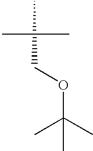 | tbutyl | tert-butyl N-(2-{4-[6- amino-5-(4- fluorobenzoyl)-2- oxopyridin-1(2H)-yl]- 3,5- difluorophenyl}ethyl)-O- tert-butyl-L-serinate | 99% purity: m/z 588 [M + H]⁺ |

### Example 33

### cyclopentyl N-(2-{4-[6-amino-5-(2,4-difluorobenzoyl)-2-oxopyridin-1(2H)-yl]-3,5-difluorophenyl}ethyl)-L-leucinate

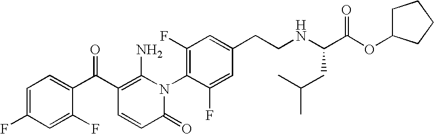

Example 33 was synthesised using Intermediate 5 as shown below.

To a solution of {4-[6-amino-5-(2,4-difluorobenzoyl)-2-oxopyridin-1(2H)-yl]-3,5-difluorophenyl}acetaldehyde (Intermediate 5) (46 mg, 0.114 mmol) in THF (2 ml) was added cyclopentyl L-leucinate (Intermediate 8) (40 mg, 0.201 mmol), stirred for 30 minutes, and then STAB (80 mg, 0.377 mmol). The reaction stirred for 24 h, diluted with EtOAc and the organic washed with sat NaHCO₃, brine, dried (MgSO₄) and concentrated in vacuo. The residue was purified by column chromatography (0.75-1.25% MeOH/DCM), and then purified by preparative HPLC to provide the title compound (29 mg, 31% yield).

LC/MS: m/z 588 [M+H]⁺. ¹H NMR (300 MHz, MeOH-d4) δ: 7.57-7.48 (2H, m), 7.32-7.10 (4H, m), 5.84 (1H, d, J=9.6 Hz), 5.41-5.30 (1H, m), 4.10-4.03 (1H, m), 3.45-3.30 (2H, m), 3.20-3.14 (2H, m), 2.05-1.60 (11H, m), 1.10-0.95 (6H, m).

The following examples were prepared as described above for Example 33 using Intermediate 5 and the appropriate amino acid ester.

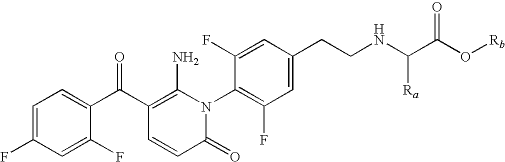

| Example | Intermediate | | | | |
|---|---|---|---|---|---|
| Number | used | Rₐ | R_{b} | Name | LCMS purity |
| 34 | 5 + 11 | 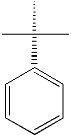 | cyclopentyl | Cyclopentyl (2S)-[(2-{4- [6-amino-5-(2,4- difluorobenzoyl)-2- oxopyridin-1(2H)-yl]- 3,5- difluorophenyl}ethyl)ami- no](phenyl)acetate | 99% purity: m/z 608 [M + H]⁺ |
| 35 | 5 | 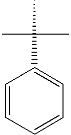 | tbutyl | tert-butyl (2S)-[(2-{4-[6- amino-5-(2,4- difluorobenzoyl)-2- oxopyridin-1(2H)-yl]- 3,5- difluorophenyl}ethyl)ami- no](phenyl)acetate | 97% purity: m/z 596 [M + H]⁺ |
| 36 | 5 + 9 | 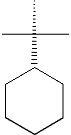 | cyclopentyl | Cyclopentyl (2S)-[(2-{4- [6-amino-5-(2,4- difluorobenzoyl)-2- oxopyridin-1(2H)-yl]- 3,5- difluorophenyl}ethyl)ami- no](cyclohexyl)acetate | 99% purity: m/z 614 [M + H]⁺ |
| 37 | 5 | 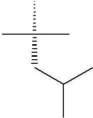 | tbutyl | tert-butyl N-(2-{4-[6- amino-5-(2,4- difluorobenzoyl)-2- oxopyridin-1(2H)-yl]- 3,5- difluorophenyl}ethyl)-L- leucinate | 99% purity: m/z 576 [M + H]⁺ |
| 38 | 5 | 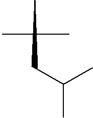 | cyclopentyl | Cyclopentyl N-(2-{4-[6- amino-5-(2,4- difluorobenzoyl)-2- oxopyridin-1(2H)-yl]- 3,5- difluorophenyl}ethyl)-D- leucinate | 99% purity: m/z 588 [M + H]⁺ |
| 39 | 5 | 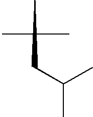 | tbutyl | tert-butyl N-(2-{4-[6- amino-5-(2,4- difluorobenzoyl)-2- oxopyridin-1(2H)-yl]- 3,5- difluorophenyl}ethyl)-D- leucinate | 99% purity: m/z 576 [M + H]⁺ |
| 40 | 5 + 15 | 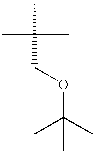 | cyclopentyl | Cyclopentyl N-(2-{4-[6- amino-5-(2,4- difluorobenzoyl)-2- oxopyridin-1(2H)-yl]- 3,5- difluorophenyl}ethyl)-O- tert-butyl-L-serinate | 98% purity: m/z 576 [M + H]⁺ |
| 41 | 5 | 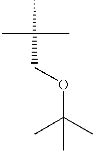 | tbutyl | tert-butyl N-(2-{4-[6- amino-5-(2,4- difluorobenzoyl)-2- oxopyridin-1(2H)-yl]- 3,5- difluorophenyl}ethyl)-O- tert-butyl-L-serinate | 99% purity: m/z 606 [M + H]⁺ |
| 42 | 5 | 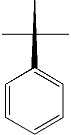 | cyclopentyl | Cyclopentyl (2R)-[(2-{4- [6-amino-5-(2,4- difluorobenzoyl)-2- oxopyridin-1(2H)-yl]- 3,5- difluorophenyl}ethyl)ami- no](phenyl)acetate | 95% purity: m/z 608 [M + H]⁺ |
| 43 | 5 + 13 | 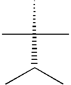 | cyclopentyl | Cyclopentyl N-[2-(4-{6- amino-5-[(2,4- difluorophenyl)carbonyl]- 2-oxopyridin-1(2H)- yl}-3,5 difluorophenyl)ethyl]-L- vainate | 99% purity: m/z 574 [M + H]⁺ |
| 44 | 5 + 18 | 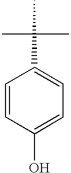 | cyclopentyl | Cyclopentyl (2S)-{[2-(4- {6-amino-5-[(2,4- difluorophenyl)carbonyl]- 2-oxopyridin-1(2H)- yl}-3,5- difluorophenyl)ethyl]ami- no}(4- hydroxyphenyl)ethanoate | 95% purity: m/z 624 [M + H]⁺ |
| 45 | 5 | 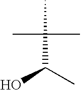 | cyclopentyl | Cyclopentyl N-[2-(4-{6- amino-5-[(2,4- difluorophenyl)carbonyl]- 2-oxopyridin-1(2H)- yl}-3,5- difluorophenyl)ethyl]-L- threoninate | 97% purity: m/z 576 [M + H]⁺ |
| 46 | 5 + 19 | 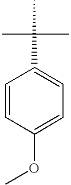 | cyclopentyl | Cyclopentyl (2S)-{[2-(4- {6-amino-5-[(2,4- difluorophenyl)carbonyl]- 2-oxopyridin-1(2H)- yl}-3,5- difluorophenyl)ethyl]ami- no}(4- methoxyphenyl)ethano ate | 95% purity: m/z 638 [M + H]⁺ |
| 47 | 5 + 21 | 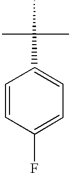 | cyclopentyl | Cyclopentyl (2S)-{[2-(4- {6-amino-5-[(2,4- difluorophenyl)carbonyl]- 2-oxopyridin-1(2H)- yl}-3,5- difluorophenyl)ethyl]ami- no}(4- fluorophenyl)ethanoate | 98% purity: m/z 626 [M + H]⁺ |
| 48 | 5 | 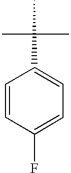 | tbutyl | tert-butyl (2S)-{[2-(4-{6- amino-5-[(2,4- difluorophenyl)carbonyl]- 2-oxopyridin-1(2H)- yl}-3,5- difluorophenyl)ethyl]ami- no}(4- fluorophenyl)ethanoate | 95% purity: m/z 614 [M + H]⁺ |

### Example 49

### Cyclopentyl N-(2-{5-[6-amino-5-(2,4-difluorobenzoyl)-2-oxopyridin-1(2H)-yl]-2-thienyl}ethyl)-L-leucinate

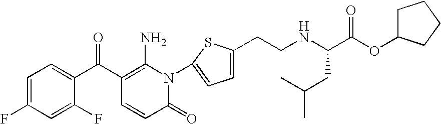

Example 49 was synthesised using Intermediate 7 and Intermediate 8.

LC/MS: m/z 558 [M+H]⁺. ¹H NMR (300 MHz, CDCl₃) δ: 10.57 (1H, br s), 7.45-7.30 (2H, m), 7.03-6.85 (4H, m), 5.86 (1H, d, J=9.6 Hz), 5.15-5.05 (1H, m), 3.24 (1H, t, J=7.2 Hz), 3.08-2.79 (4H, m), 2.00-1.60 (9H, m), 1.43 (2H, t, J=7.2 Hz), 0.95-0.88 (6H, m).

### Example 50

### tbutyl N-(2-{5-[6-amino-5-(2,4-difluorobenzoyl)-2-oxopyridin-1(2H)-yl]-2-thienyl}ethyl)-L-leucinate

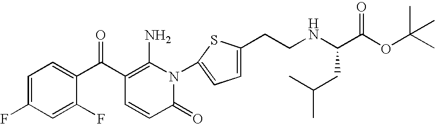

Example 50 was synthesised using Intermediate 7 and tbutyl-L-leucinate.

LC/MS: m/z 546 [M+H]⁺. ¹H NMR (300 MHz, CDCl₃) δ: 10.60 (1H, br s), 7.47-7.30 (2H, m), 7.08-6.85 (4H, m), 5.89 (1H, d, J=9.6 Hz), 3.18 (1H, t, J=7.4 Hz), 3.05-2.80 (4H, m), 1.80-1.69 (1H, m), 1.49 (9H, s), 1.48-1.35 (2H, m), 0.97-0.91 (6H, m).

### NMR Data of all Ester Derivatives Described Above

| Example | |
|---|---|
| Number | ¹H NMR assignments |
| 2 | (DMSO-d6) 10.17-9.83 (3H, m), 7.56-7.21 (13H, m), 5.71 (1H, d, J = 9.6 Hz), |
| | 5.24 (1H, m), 3.11-2.93 (4H, m), 1.39 (9H, s) |
| 3 | (DMSO-d6) 7.52-7.24 (8H, m), 5.71 (1H, d, J = 9.6 Hz), 5.18 (1H, m), |
| | 2.91-2.73 (4H, m), 1.84-1.64 (14H, m), 1.18-1.10 (6H, m) |
| 4 | (DMSO-d6) 9.38-9.28 (2H, m), 7.55-7.20 (8H, m), 5.72 (1H, d, J = 9.9 Hz), |
| | 3.91 (1H, m), 3.14 (3H, m), 2.05 (1H, m), 1.91-1.57 (5H, m), 1.51 (9H, s), |
| | 1.44-0.93 (6H, m) |
| 5 | (DMSO-d6) 10.07 (1H, br s), 9.44 (2H, m), 7.51-7.25 (8H, m), 5.72 (1H, d, |
| | J = 9.9 Hz), 5.28 (1H, m), 4.06 (1H, m), 3.15 (4H, m), 2.49 (1H, m), |
| | 1.88-1.65 (8H, m), 1.09-0.97 (6H, m) |
| 6 | (DMSO-d6) 9.39-9.27 (2H, m), 7.51-7.25 (8H, m), 5.72 (1H, d, J = 9.9 Hz), |
| | 3.98 (1H, m), 3.15 (4H, m), 2.49 (1H, m), 1.51 (9H, s), 1.10 (3H, d, J = 6.6 Hz), |
| | 0.99 (3H, d, J = 6.6 Hz) |
| 7 | (DMSO-d6) 7.55-7.38 (4H, m), 7.27-7.20 (4H, m), 5.71 (1H, d, J = 9.9 Hz), |
| | 5.15 (1H, m), 2.87-2.64 (5H, m), 1.84-1.57 (8H, m), 0.93 (9H, s) |
| 8 | (DMSO-d6) 7.55-7.24 (8H, m), 5.72 (1H, d, J = 9.6 Hz), 3.33-2.88 (4H, m), |
| | 1.49 (9H, s), 1.12-1.02 (10H, m) |
| 9 | (DMSO-d6) 7.51-7.21 (8H, m), 5.71 (1H, d, J = 9.6 Hz), 5.25 (1H, m), 4.04 (1H, |
| | m), 3.27-3.10 (4H, m), 1.91-1.65 (11H, m), 0.93 (6H, d, J = 4.5 Hz) |
| 10 | (DMSO-d6) 10.07 (1H, br s), 9.58-9.51 (2H, m), 7.55-7.21 (8H, m), 5.72 (1H, |
| | d, J = 9.9 Hz), 3.92 (1H, m), 3.12 (4H, m), 1.76 (3H, m), 1.49 (9H, s), 0.95 (6H, |
| | d, J = 4.2 Hz) |
| 11 | (DMSO-d6) 7.55-7.37 (4H, m), 7.26-7.20 (4H, m), 5.71 (1H, d, J = 9.6 Hz), |
| | 5.12 (1H, m), 3.53-3.36 (2H, m), 2.89-2.78 (4H, m), 2.03 (1H, m), 1.82 (2H, m), |
| | 1.66-1.56 (6H, m), 1.10 (9H, s) |
| 12 | (DMSO-d6) 10.06 (1H, br s), 7.55-7.37 (4H, m), 7.27-7.20 (4H, m), 5.71 (1H, |
| | d, J = 9.6 Hz), 3.50 (1H, m), 3.44 (1H, m), 3.26 (1H, m), 2.91-2.78 (4H, m), |
| | 1.43 (9H, s), 1.11 (9H, s) |
| 13 | (CDCl₃) 7.50-7.34 (4H, m), 7.25 (2H, d, J = 8.5 Hz), 7.02 (1H, td, J = 2.3, 8.5 Hz), |
| | 6.94 (1H, td, J = 2.3, 9.2 Hz), 5.92 (1H, d, J = 9.8 Hz), 4.73 (1H, td, J = 4.3, |
| | 10.9 Hz), 3.28 (1H, t, J = 7.4 Hz), 2.96-2.79 (4H, m), 1.99-1.85 (2H, m), |
| | 1.76-1.65 (3H, m), 1.59-1.37 (6H, m), 1.13-0.88 (12H, m), 0.77 (3H, d, J = 7.0 Hz) |
| 14 | (CDCl₃) 7.50-7.41 (3H, m), 7.36 (1H, dd, J = 3.0, 9.8 Hz), 7.27-7.24 (2H, m), |
| | 7.02 (1H, td, J = 2.3, 8.5 Hz), 6.94 (1H, td, J = 2.3, 9.2 Hz), 5.93 (1H, d, J = 9.8 Hz), |
| | 4.69 (1H, td, J = 4.4, 10.9 Hz), 3.30-3.23 (1H, m), 2.97-2.80 (4H, m), |
| | 2.09-2.00 (1H, m), 1.94-1.87 (1H, m), 1.78-1.68 (4H, m), 1.55-1.38 (5H, m), |
| | 1.09-0.88 (12H, m), 0.77 (3H, d, J = 7.0 Hz) |
| 15 | (CDCl₃) 7.30-7.52 (4H, m), 7.24 (2H, d, J = 8.5 Hz), 6.88-7.05 (2H, m), 5.91 (1H, |
| | d, J = 9.6 Hz), 5.14-5.21 (1H, m), 3.86-3.95 (1H, m), 3.10 (1H, d, J = 4.7 Hz), |
| | 2.72-2.98 (4H, m), 2.0 (2H, s), 1.57-1.94 (8H, m), 1.11-1.22 (11H, m) |
| 16 | (CDCl₃) 7.32-7.52 (4H, m), 7.24 (2H, d, J = 8.5 Hz), 6.88-7.05 (2H, m), |
| | 5.92 (1H, d, J = 9.8 Hz), 3.81-3.91 (1H, m, J = 6.2, 6.0, 5.9, 5.9 Hz), 3.44 (2H, br. s.), |
| | 3.07 (1H, d, J = 5.3 Hz), 2.80-2.97 (4H, m), 1.48 (9H, s), 1.12-1.20 (12H, m) |
| 17 | (CDCl₃) 7.31-7.50 (4H, m), 7.25 (2H, br. s.), 6.88-7.04 (2H, m), 5.91 (1H, d, |
| | J = 9.8 Hz), 5.17-5.25 (1H, m), 3.54 (1H, dd, J = 7.9, 6.2 Hz), 2.98-3.10 (1H, |
| | m), 2.80-2.97 (4H, m), 1.82-1.95 (1H, m), 1.57-1.77 (7H, m), 1.17 (3H, d, |
| | J = 6 Hz) |
| 18 | (CDCl₃) 7.32-7.51 (4H, m), 7.24 (1H, d, J = 8.7 Hz), 6.88-7.05 (2H, m), |
| | 5.92 (1H, d, J = 9.8 Hz), 5.15-5.22 (1H, m), 3.06 (1H, d, J = 5.8 Hz), 2.77-2.97 (4H, |
| | m), 1.80-1.94 (1H, m), 1.57-1.77 (6H, m), 1.44-1.56 (1H, m), 1.11-1.27 (1H, m), |
| | 0.85-0.94 (6H, m) |
| 19 | (CDCl₃) 7.30-7.53 (4H, m), 7.26 (2H, d, J = 4.1 Hz), 6.87-7.05 (2H, m), |
| | 5.92 (1H, d, J = 9.8 Hz), 2.78-3.03 (7H, m), 2.06 (1H, s), 1.58-1.72 (1H, m), |
| | 1.47 (9H, s), 1.12-1.27 (1H, m), 0.83-0.94 (5H, m) |
| 20 | (DMSO-d6) 7.55-7.10 (8H, m), 5.72 (1H, d, J = 9.6 Hz), 5.20 (1H, m), 3.95 (1H, |
| | m), 3.15-3.01 (4H, m), 1.86-1.40 (11H, m) |
| 21 | (DMSO-d6) 7.55-7.37 (4H, m), 7.27-7.21 (4H, m), 5.71 (1H, d, J = 9.6 Hz), |
| | 3.21 (1H, q, J = 6.9 Hz), 2.86-2.72 (4H, m), 1.50 (9H, s), 1.16 (3H, d, J = 6.9 Hz) |
| 22 | (DMSO-d6) 7.56-7.19 (13H, m), 5.72 (1H, d, J = 9.9 Hz), 5.01 (1H, m), 3.49 (1H, |
| | m), 2.95-2.74 (6H, m), 1.76-1.32 (8H, m) |
| 23 | (DMSO-d6) 7.55-7.37 (4H, m), 7.31-7.21 (9H, m), 5.72 (1H, d, J = 9.9 Hz), |
| | 3.40 (1H, t, J = 7.5 Hz), 2.93-2.73 (6H, m), 1.29 (9H, s) |
| 26 | (CDCl₃) 7.64-7.55 (3H, m), 7.43-7.25 (5H, m), 7.17 (2H, t, J = 8.6 Hz), 7.04 (2H, |
| | d, J = 8.4 Hz), 5.89 (1H, d, J = 9.6 Hz), 5.20-5.14 (1H, m), 4.3 (1H, s), |
| | 2.95-2.80 (4H, m), 2.05-1.45 (8H, m) |
| 27 | (CDCl₃) 7.64-7.55 (3H, m), 7.40-7.29 (5H, m), 7.17 (2H, t, J = 8.6 Hz), 7.03 (2H, |
| | d, J = 7.8 Hz), 5.88 (1H, d, J = 9.9 Hz), 4.26 (1H, s), 2.92-2.86 (4H, m), 1.40 (9H, |
| | s) |
| 28 | (MeOH-d4) 7.74 (1H, d, J = 9.9 Hz), 7.67-7.60 (2H, m), 7.30-7.20 (4H, m), |
| | 5.83 (1H, d, J = 9.9 Hz), 5.41-5.30 (1H, m), 4.00-3.90 (1H, m), 3.22-3.14 (2H, m), |
| | 2.10-1.60 (14H, m), 1.45-0.95 (5H, m) |
| 29 | (MeOH-d4) 7.74 (1H, d, J = 9.6 Hz), 7.66-7.60 (2H, m), 7.32-7.23 (4H, m), |
| | 5.84 (1H, d, J = 9.6 Hz), 5.40-5.34 (1H, m), 4.10-4.03 (1H, m), 3.47-3.32 (2H, m), |
| | 3.21-3.15 (2H, m), 2.05-1.60 (11H, m), 1.04 (6H, t, J = 5.7 Hz) |
| 30 | (MeOH-d4) 7.74 (1H, d, J = 9.6 Hz), 7.66-7.61 (2H, m), 7.32-7.20 (4H, m), |
| | 5.84 (1H, d, J = 9.6 Hz), 4.03-3.96 (1H, m), 3.47-3.32 (2H, m), 3.22-3.14 (2H, m), |
| | 1.89-1.60 (3H, m), 1.57 (9H, s), 1.06 (6H, t, J = 5.9 Hz) |
| 31 | (MeOH-d4) 7.74 (1H, d, J = 9.9 Hz), 7.66-7.60 (2H, m), 7.29-7.22 (4H, m), |
| | 5.83 (1H, d, J = 9.9 Hz), 5.40-5.30 (1H, m), 4.35-4.30 (1H, m), 4.01-3.88 (2H, m), |
| | 3.48-3.38 (2H, m), 3.22-3.17 (2H, m), 2.05-1.60 (8H, m), 1.26 (9H, s) |
| 32 | (MeOH-d4) 7.74 (1H, d, J = 9.6 Hz), 7.66-7.61 (2H, m), 7.31-7.23 (4H, m), |
| | 5.84 (1H, d, J = 9.9 Hz), 4.26-4.22 (1H, m), 4.01-3.86 (2H, m), 3.46-3.36 (2H, m), |
| | 3.27-3.18 (2H, m), 1.57 (9H, s), 1.28 (9H, s) |
| 34 | (MeOH-d4) 7.59-7.47 (7H, m), 7.25-7.10 (4H, m), 5.83 (1H, d, J = 9.6 Hz), |
| | 5.40-5.25 (1H, m), 5.19 (1H, s), 3.25-3.05 (3H, m), 2.00-1.70 (3H, m), |
| | 1.65-1.30 (5H, m) |
| 35 | (MeOH-d4) 7.59-7.47 (7H, m), 7.24-7.10 (4H, m), 5.83 (1H, d, J = 9.9 Hz), |
| | 5.14 (1H, s), 3.25-3.05 (3H, m), 1.46 (9H, s) |
| 36 | (MeOH-d4) 7.57-7.48 (2H, m), 7.30-7.10 (4H, m), 5.84 (1H, d, J = 9.9 Hz), |
| | 5.40-5.30 (1H, m), 4.00-3.90 (1H, m), 3.20-3.10 (2H, m), 2.10-1.60 (15H, m), |
| | 1.45-1.00 (6H, m) |
| 37 | (MeOH-d4) 7.57-7.48 (2H, m), 7.29 (2H, d, J = 8.7 Hz), 7.20-7.05 (2H, m), |
| | 5.84 (1H, d, J = 9.9 Hz), 4.02-3.96 (1H, m), 3.46-3.35 (2H, m), 3.21-3.14 (2H, m), |
| | 1.90-1.65 (3H, m), 1.57 (9H, s), 1.06 (6H, t, J = 5.8 Hz) |
| 38 | (MeOH-d4) 7.57-7.48 (2H, m), 7.29 (2H, d, J = 8.7 Hz), 7.14 (2H, t, J = 8.5 Hz), |
| | 5.83 (1H, d, J = 9.6 Hz), 5.40-5.34 (1H, m), 4.10-4.02 (1H, m), 3.50-3.32 (2H, |
| | m), 3.21-3.15 (2H, m), 2.02-1.60 (11H, m), 1.04 (6H, t, J = 5.9 Hz) |
| 39 | (MeOH-d4) 7.57-7.48 (2H, m), 7.29 (2H, d, J = 8.7 Hz), 7.18-7.10 (2H, m), |
| | 5.84 (1H, d, J = 9.9 Hz), 4.02-3.86 (1H, m), 3.47-3.32 (2H, m), 3.22-3.14 (2H, m), |
| | 1.89-1.73 (11H, m), 1.57 (9H, s), 1.05 (6H, t, J = 5.9 Hz) |
| 40 | (MeOH-d4) 7.57-7.48 (2H, m), 7.29 (2H, d, J = 8.4 Hz), 7.18-7.09 (2H, m), |
| | 5.84 (1H, d, J = 9.6 Hz), 5.43-5.30 (1H, m), 4.35-4.30 (1H, m), 4.02-3.86 (2H, m), |
| | 3.48-3.35 (2H, m), 3.25-3.18 (2H, m), 2.05-1.65 (8H, m), 1.26 (9H, s) |
| 41 | (MeOH-d4) 7.55-7.48 (2H, m), 7.29 (2H, d, J = 8.7 Hz), 7.18-7.10 (2H, m), |
| | 5.84 (1H, d, J = 9.6 Hz), 4.30-4.20 (1H, m), 4.01-3.86 (2H, m), 3.46-3.36 (2H, m), |
| | 3.27-3.18 (2H, m), 1.57 (9H, s), 1.28 (9H, s) |
| 42 | (CDCl₃) 7.28-7.41 (4 H, m), 7.18-7.21 (3 H, m), 6.82-7.01 (4 H, m), 5.83 (1 |
| | H, d, J = 9.8 Hz), 5.05-5.14 (1 H, m), 4.25 (1 H, s), 2.75-2.89 (4 H, m), |
| | 1.36-1.83 (8 H, m) |
| 43 | (CDCl₃) 7.48-7.36 (2H, m), 7.11-6.90 (4H, m), 5.90 (1H, d, J = 9.9 Hz), |
| | 5.22-5.15 (1H, m), 2.95-2.75 (5H, m), 2.00-1.50 (9H, m), 0.95-0.91 (6H, m). |
| 44 | (MeOH-d4) 7.44-7.55 (2H, m), 7.32 (2H, d, J = 8.5 Hz), 7.06-7.24 (4H, m), |
| | 6.90 (2H, d, J = 8.5 Hz), 5.81 (1H, d, J = 9.6 Hz), 5.27-5.35 (1H, m), 5.08 (1H, |
| | s), 3.04-3.28 (4H, m), 1.40-1.95 (8H, m) |
| 45 | (MeOH-d4) 7.46-7.56 (2H, m), 7.26 (2H, d, J = 9.0 Hz), 7.12 (2H, t, J = 8.5 Hz), |
| | 5.82 (1H, d, J = 9.6 Hz), 5.36 (1H, t, J = 5.4 Hz), 4.12-4.21 (1H, m), |
| | 3.91-3.97 (1H, m), 3.34-3.50 (2H, m), 3.06-3.25 (2H, m), 1.64-2.04 (8H, m) |
| 46 | (MeOH-d4) 7.39-7.55 (4H, m), 7.04-7.24 (6H, m), 5.81 (1H, d, J = 9.6 Hz), |
| | 5.29-5.36 (1H, m), 5.14 (1H, s), 3.84 (3H, s), 3.35 (2H, d), 3.06-3.25 (3H, |
| | m), 1.44-1.95 (8H, m) |
| 47 | (CDCl₃) 7.23-7.49 (5H, m), 6.88-7.10 (6H, m), 5.89 (1H, d, J = 9.8 Hz), |
| | 5.12-5.21 (1H, m), 4.30 (1H, s), 2.80-2.97 (4H, m), 1.38-2.02 (8H, m) |
| 48 | (CDCl₃) 7.45-7.29 (4H, m), 7.07-6.87 (6H, m), 5.86 (1H, d, J = 9.6 Hz), 4.23 (1H, |
| | s), 2.90-2.78 (4H, m), 1.38 (9H, s) |

### Example 51

### (2S)-[(2-{4-[6-amino-5-(2,4-difluorobenzoyl)-2-oxopyridin-1(2H)yl]-phenyl}ethyl)amino](phenyl)acetic acid

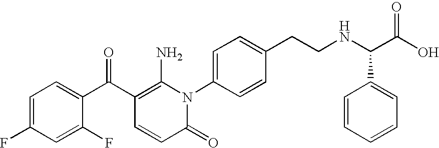

To a solution of tert-butyl (2S)-{[2-(4-{6-amino-5-[(2,4-difluorophenyl)carbonyl]-2-oxopyridin-1(2H)-yl}phenyl)ethyl]amino}(phenyl)ethanoate (Example 2) (30 mg, 0.05 mmol) in DCM (2 ml) was added trifluoroacetic acid (2 ml). The mixture was stirred at room temperature for 16 hrs and concentrated under reduced pressure. The residue was triturated with Et₂O, collected by filtration and dried under reduced pressure to afford the title compound as a brown solid as a di-TFA salt (21 mg, 51% yield).

LC/MS: m/z 504 [M+H]⁺. ¹H NMR (300 MHz, DMSO-d₆) δ: 7.52-7.23 (13H, m), 5.71 (1H, d, J=9.9 Hz), 4.42 (1H, m), 3.02 (4H, m).

The following examples were all prepared in a similar manner to Example 51. Where necessary, the compounds were purified by preparative HPLC to achieve good purity.

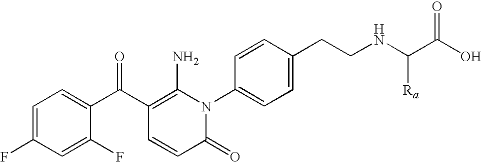

| Example | Example | | | |
|---|---|---|---|---|
| Number | used | Rₐ | Name | LCMS purity |
| 52 | 4 | 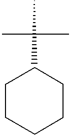 | (2S)-{[2-(4-{6-amino-5- [(2,4- difluorophenyl)carbonyl]- 2-oxopyridin-1(2H)- yl}phenyl)ethyl]amino}(cyclo- hexyl)ethanoic acid | 100% purity: m/z 510 [M + H]⁺ |
| 53 | 6 | 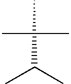 | N-[2-(4-{6-amino-5- [(2,4- difluorophenyl)carbonyl]- 2-oxopyridin-1(2H)- yl}phenyl)ethyl]-L- valine | 91% purity: m/z 470 [M + H]⁺ |
| 54 | 8 | 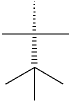 | N-[2-(4-{6-amino-5- [(2,4- difluorophenyl)carbonyl]- 2-oxopyridin-1(2H)- yl}phenyl)ethyl]-3- methyl-L-valine | 95% purity: m/z 484 [M + H]⁺ |
| 55 | 10 | 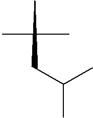 | N-[2-(4-{6-amino-5- [(2,4- difluorophenyl)carbonyl]- 2-oxopyridin-1(2H)- yl}phenyl)ethyl]-D- leucine | 92% purity: m/z 484 [M + H]⁺ |
| 56 | Intermediate 2 + tbutyl-L- serine | 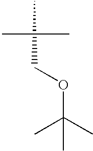 | N-[2-(4-{6-amino-5- [(2,4- difluorophenyl)carbonyl]- 2-oxopyridin-1(2H)- yl}phenyl)ethyl]-O-tert- butyl-L-serine | 99% purity: m/z 514 [M + H]⁺ |
| 57 | 12 | 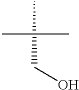 | N-[2-(4-{6-amino-5- [(2,4- difluorophenyl)carbonyl]- 2-oxopyridin-1(2H)- yl}phenyl)ethyl]-L- serine | 90% purity: m/z 458 [M + H]⁺ |
| 58 | 16 | 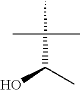 | N-[2-(4-{6-amino-5- [(2,4- difluorophenyl)carbonyl]- 2-oxopyridin-1(2H)- yl}phenyl)ethyl]-L- threonine | 98% purity: m/z 472 [M + H]⁺ |
| 59 | 19 | 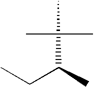 | N-[2-(4-{6-amino-5- [(2,4- difluorophenyl)carbonyl]- 2-oxopyridin-1(2H)- yl}phenyl)ethyl]-L- isoleucine | 95% purity: m/z 484 [M + H]⁺ |
| 60 | 21 | 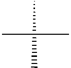 | N-[2-(4-{6-amino-5- [(2,4- difluorophenyl)carbonyl]- 2-oxopyridin-1(2H)- yl}phenyl)ethyl]-L- alanine | 99% purity: m/z 442 [M + H]⁺ |
| 61 | 23 | 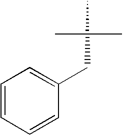 | N-[2-(4-{6-amino-5- [(2,4- difluorophenyl)carbonyl]- 2-oxopyridin-1(2H)- yl}phenyl)ethyl]-L- phenylalanine | 96% purity: m/z 518 [M + H]⁺ |

The following examples were all prepared in a similar manner to Example 51. Where necessary, the compounds were purified by preparative HPLC to achieve good purity.

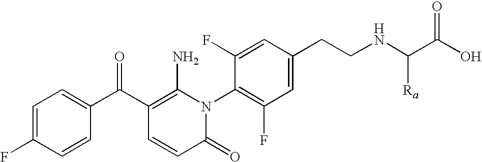

| Example | Example | | | |
|---|---|---|---|---|
| Number | used | Rₐ | Name | LCMS purity |
| 62 | 27 | 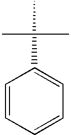 | (2S)-[(2-{4-[6-amino-5- (4-fluorobenzoyl)-2- oxopyridin-1(2H)-yl]- 3,5- difluorophenyl}ethyl)ami- no](phenyl)acetic acid | 99% purity: m/z 522 [M + H]⁺ |
| 63 | 25 | 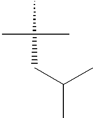 | N-(2-{4-[6-amino-5-(4- fluorobenzoyl)-2- oxopyridin-1(2H)-yl]- 3,5- difluorophenyl}ethyl)-L- leucine | 99% purity: m/z 502 [M + H]⁺ |
| 64 | 30 | 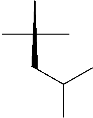 | N-(2-{4-[6-amino-5-(4- fluorobenzoyl)-2- oxopyridin-1(2H)-yl]- 3,5- difluorophenyl}ethyl)-D- leucine | 95% purity: m/z 502 [M + H]⁺ |
| 65 | Intermediate 4b + tbutyl-L- serine | 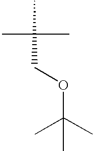 | N-(2-{4-[6-amino-5-(4- fluorobenzoyl)-2- oxopyridin-1(2H)-yl]- 3,5- difluorophenyl}ethyl)-O- tert-butyl-L-serine | 95% purity: m/z 532 [M + H]⁺ |

The following examples were all prepared in a similar manner to Example 51. Where necessary, the compounds were purified by preparative HPLC to achieve good purity.

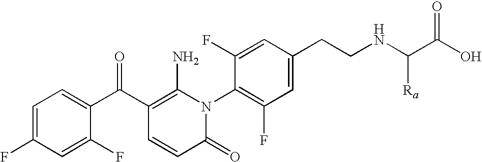

| Example | Example | | | |
|---|---|---|---|---|
| Number | used | Rₐ | Name | LCMS purity |
| 66 | 35 | 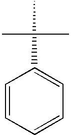 | (2S)-[(2-{4-[6-amino-5- (2,4-difluorobenzoyl)-2- oxopyridin-1(2H)-yl]- 3,5- difluorophenyl}ethyl)ami- no](phenyl)acetic acid | 97% purity: m/z 540 [M + H]⁺ |
| 67 | 37 | 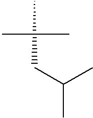 | N-(2-{4-[6-amino-5- (2,4-difluorobenzoyl)-2- oxopyridin-1(2H)-yl]- 3,5- difluorophenyl}ethyl)-L- leucine | 99% purity: m/z 520 [M + H]⁺ |
| 68 | 39 | 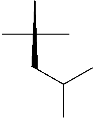 | N-(2-{4-[6-amino-5- (2,4-difluorobenzoyl)-2- oxopyridin-1(2H)-yl]- 3,5- difluorophenyl}ethyl)-D- leucine | 98% purity: m/z 520 [M + H]⁺ |
| 69 | Intermediate 5 + tbutyl-L- serine | 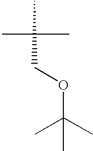 | N-(2-{4-[6-amino-5- (2,4-difluorobenzoyl)-2- oxopyridin-1(2H)-yl]- 3,5- difluorophenyl}ethyl)-O- tert-butyl-L-serine | 93% purity: m/z 550 [M + H]⁺ |
| 70 | 48 | 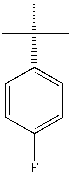 | (2S)-{[2-(4-{6-amino-5- [(2,4- difluorophenyl)carbonyl]- 2-oxopyridin-1(2H)- yl}-3,5- difluorophenyl)ethyl]ami- no}(4- fluorophenyl)ethanoic acid | 95% purity: m/z 558 [M + H]⁺ |

### Example 71

### N-(2-{5-[6-amino-5-(2,4-difluorobenzoyl)-2-oxopyridin-1(2H)-yl]-2-thienyl}ethyl)-L-leucine

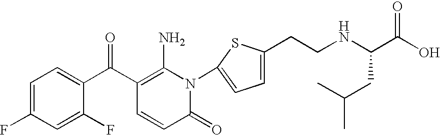

From Example 50. LC/MS: m/z 490 [M+H]⁺. ¹H NMR (300 MHz, DMSO-d₆) δ: 10.22 (1H, br s), 7.56-7.35 (2H, m), 7.30-7.15 (2H, m), 7.10-6.95 (2H, m), 5.69 (1H, d, J=9.9 Hz), 3.35-3.20 (1H, m), 3.15-2.95 (4H, m), 1.85-1.70 (1H, m), 1.60-1.35 (2H, m), 0.90 (6H, t, J=5.9 Hz)

### NMR Data of all Acid Derivatives Described Above

| Example | |
|---|---|
| Number | ¹H NMR assignments |
| 52 | (DMSO-d6) 10.00 (1H, br s), 9.04 (2H, m), 7.46-7.12 (8H, m), 6.73 (1H, br s), |
| | 5.65 (1H, d, J = 9.6 Hz), 3.91 (1H, m), 3.19-2.98 (4H, m), 2.33 (1H, m), |
| | 0.96 (6H, m) |
| 53 | (DMSO-d6) 7.55-7.21 (8H, m), 5.72 (1H, d, J = 9.9 Hz), 3.49 (1H, br s), |
| | 3.09 (4H, m), 1.06 (9H, s) |
| 54 | (DMSO-d6) 10.11 (1H, br s), 9.16 (2H, br s), 7.55-7.21 (8H, m), 6.89 (1H, br |
| | s), 5.72 (1H, d, J = 9.9 Hz), 4.00 (1H, m), 3.05-2.89 (4H, m), 1.73 (3H, m), |
| | 0.95 (6H, d, J = 5.7 Hz) |
| 55 | (DMSO-d6) 10.02 (1H, br s), 7.48-7.24 (8H, m), 5.72 (1H, d, J = 9.6 Hz), |
| | 3.71 (1H, m), 3.57-3.01 (6H, m), 1.16 (9H, s) |
| 56 | (DMSO-d6) 10.07 (1H, br s), 9.14 (2H, br s), 7.55-7.21 (8H, m), 6.91 (1H, br |
| | s), 5.72 (1H, d, 9.9 Hz), 4.15 (1H, s), 3.97 (2H, m), 3.17-3.10 (4H, m) |
| 57 | (DMSO-d6) 10.04 (1H, br s.), 7.35-7.56 (4H, m), 7.18-7.34 (4H, m), |
| | 5.71 (1H, d, J = 9.8 Hz), 3.86-3.96 (1H, m), 3.07-3.20 (3H, m), 2.96-3.07 (3H, m), |
| | 1.19 (3H, d, J = 6.2 Hz) |
| 58 | (DMSO-d6) 7.35-7.58 (5H, m), 7.17-7.33 (4H, m), 5.71 (1H, d, J = 10.0 Hz), |
| | 2.92-3.14 (4H, m), 1.73-1.88 (1H, m), 1.49 1.63 (1H, m), 1.17-1.38 (1H, m), |
| | 0.81-0.96 (5H, m) |
| 59 | (DMSO-d6) 7.50-7.28 (8H, m), 5.72 (1H, d, J = 9.9 Hz), 3.45-3.00 (5H, m), |
| | 1.34 (3H, d, J = 7.0 Hz) |
| 60 | (DMSO-d6) 7.51-7.23 (13H, m), 5.71 (1H, d, J = 9.6 Hz), 3.63 (1H, m), |
| | 2.98-2.90 (6H, m) |
| 61 | (DMSO-d6) 7.61-7.50 (3H, m), 7.46-7.25 (9H, m), 5.73 (1H, d, J = 9.9 Hz), |
| | 4.38 (1H, s), 3.10-2.80 (4H, m) |
| 62 | (MeOH-d4) 7.73 (1H, d, J = 9.9 Hz), 7.70-7.60 (2H, m), 7.31-7.20 (4H, m), |
| | 5.83 (1H, d, J = 9.6 Hz), 3.77-3.70 (1H, m), 3.20-3.05 (2H, m), 1.95-1.60 (3H, m), |
| | 1.07-1.00 (6H, m) |
| 63 | (MeOH-d4) 7.75 (1H, d, J = 9.6 Hz), 7.66-7.60 (2H, m), 7.31-7.20 (4H, m), |
| | 5.84 (1H, d, J = 9.9 Hz), 3.96-3.91 (1H, m), 3.48-3.35 (2H, m), 3.22-3.15 (2H, m), |
| | 2.00-1.65 (3H, m), 1.05 (6H, t, J = 6.2 Hz) |
| 64 | (DMSO-d6) 7.63-7.54 (3H, m), 7.38-7.32 (4H, m), 5.74 (1H, d, J = 10.8 Hz), |
| | 4.12 (1H, m), 3.80 (2H, m), 3.28 (2H, m), 3.13 (2H, m), 1.18 (9H, s) |
| 65 | (DMSO-d6) 7.62-7.53 (1H, m), 7.46-7.21 (10H, m), 5.74 (1H, d, J = 9.6 Hz), |
| | 4.36 (1H, s), 3.10-2.80 (4H, m) |
| 66 | (MeOH-d4) 7.57-7.48 (2H, m), 7.29 (2H, d, J = 9 Hz), 7.18-7.10 (2H, m), |
| | 5.84 (1H, d, J = 9.6 Hz), 3.95-3.80 (1H, m), 3.46-3.35 (2H, m), 3.21-3.10 (2H, m), |
| | 1.90-1.6 (3H, m), 1.05 (6H, t, J = 6.3 Hz) |
| 67 | (MeOH-d4) 7.57-7.48 (2H, m), 7.32-7.14 (4H, m), 5.84 (1H, d, J = 9.6 Hz), |
| | 3.95-3.85 (1H, m), 3.45-3.32 (2H, m), 3.21-3.15 (2H, m), 1.95-1.65 (3H, m), |
| | 1.05 (6H, t, J = 6.2 Hz) |
| 68 | (DMSO-d6) 7.62-7.54 (1H, m), 7.45-7.22 (5H, m), 5.75 (1H, d, J = 9.6 Hz), |
| | 3.77-3.69 (3H, m), 3.20 (2H, m), 3.08 (2H, m), 1.17 (9H, s) |
| 69 | (DMSO-d6) 7.62-7.54 (1H, m), 7.45-7.22 (5H, m), 5.75 (1H, d, J = 9.6 Hz), |
| | 3.77-3.69 (3H, m), 3.20 (2H, m), 3.08 (2H, m), 1.17 (9H, s) |
| 70 | (DMSO-d6) 7.62-7.20 (10H, m), 5.75 (1H, d, J = 9.9 Hz), 4.74 (1H, br s), |
| | 3.15-2.90 (4H, m) |

### Measurement of Biological Activities

### p38 MAP Kinase Activity

The ability of compounds to inhibit p38 MAP a Kinase activity was measured in an assay performed by Upstate (Dundee UK). In a final reaction volume of 25 μL, p38 MAP Kinase a (5-10 mU) is incubated with 25 mM Tris pH 7.5, 0.002 mMEGTA, 0.33 mg/mL myelin basic protein, 10 mM MgAcetate and [g-33P-ATP] (specific activity approx. 500 cpm/pmol, concentration as required). The reaction is initiated by the addition of the MgATP mix. After incubation for 40 minutes at room temperature, the reaction is stopped by the addition of 5 μL of a 3% phosphoric acid solution. 10 μL of the reaction is then spotted onto a P30 filtermat and washed three times for 5 minutes in 75 mM phosphoric acid and once in methanol prior to drying and scintillation counting.

Duplicate data points are generated from a 1/3 log dilution series of a stock solution in DMSO. Nine dilutions steps are made from a top concentration of 10 μM, and a ‘no compound’ blank is included. The standard radiometric filter-binding assay is performed at an ATP concentration at, or close to, the Km. Data from scintillation counts are collected and subjected to free-fit analysis by Prism software. From the curve generated, the concentration giving 50% inhibition is determined and reported.

### LPS-Stimulation of THP-1 Cells

THP-1 cells were plated in 100 μl at a density of 4×10⁴ cells/well in V-bottomed 96 well tissue culture treated plates and incubated at 37° C. in 5% CO₂ for 16 hrs. 2 hrs after the addition of the inhibitor in 100 μl of tissue culture media, the cells were stimulated with LPS (E coli strain 005:B5, Sigma) at a final concentration of 1 μg/ml and incubated at 37° C. in 5% CO₂ for 6 hrs. TNF-α levels were measured from cell-free supernatants by sandwich ELISA (R&D Systems #QTA00B).

### Cellular Assay for the Phosphorylation of MAPKAP-2 by P38

U937 or HUT78 cells were plated in RPMI 1640, and were incubated at 37° C., 5% CO₂ for 18 hours. 10 mM stocks of compounds were diluted media/0.1% DMSO to give a log or semi-log dilution series. The wells for ‘no treatment’ and ‘anisomycin’ were treated with 0.1% DMSO only. The cells were incubated at 37° C., 5% CO₂ for a further 4 hours. Anisomycin was added to all wells except ‘no treatment’ at a final concentration of 10 μM. The cells were incubated at 37° C., 5% CO₂ for 30 minutes before harvest. Plates were stood on ice whilst harvesting, and all the following steps were carried out at 4° C. The cells were pelleted at 1000 rpm for 10 minutes at 4° C., the media aspirated, and the pellet washed with cold PBS. The pellets were lysed in 120 μl of SDS lysis buffer (62.5 mM Tris, pH 6.8, 2% SDS, 10% glycerol, 50 mM DTT, with protease inhibitors and phosphatase inhibitors added according to the manufacturers' recommendations). After 30 minutes on ice, the samples were sonicated for 5 seconds before centrifugation at 13,000 rpm 4° C. for 10 minutes to remove cell debris. 10 μl of the resulting gel samples were loaded per lane on NOVEX 4-12% Bis-Tris MOPS gels. Membranes from western transfer of gels were blotted with anti-phospho MAPKAP-2, anti-phospho HSP27 and anti-GAPDH according to the manufacturers' instructions. Signal was visualised using HRP-linked anti-rabbit or anti-mouse antibodies, ECL reagent and ECL film. IC50 values for the various compounds were visualised from the resulting photographic images, using both band-shift and signal intensity.

IC50 values were allocated to one of three ranges as follows:

### Range A: IC50<100 nM

Range B: 100 nM<IC50<1000 nM

### Range C: IC50>1000 nM

### Results Table

| | | Inhibitor activity |
|---|---|---|
| | Inhibitor activity | versus THP-1 TNFα |
| Example | versus p38 MAPKa | release |
| 1 | B | A |
| 2 | B | NT |
| 3 | B | NT |
| 4 | C | NT |
| 5 | B | A |
| 6 | B | B |
| 7 | B | C |
| 8 | B | C |
| 9 | B | B |
| 10 | B | NT |
| 11 | B | A |
| 12 | B | C |
| 13 | C | A |
| 14 | C | NT |
| 15 | B | C |
| 16 | B | NT |
| 17 | A | A |
| 18 | B | A |
| 19 | B | NT |
| 20 | A | A |
| 21 | A | NT |
| 22 | B | A |
| 23 | B | NT |
| 24 | A | A |
| 25 | B | A |
| 26 | B | A |
| 27 | B | A |
| 28 | B | B |
| 29 | B | A |
| 30 | B | B |
| 31 | B | A |
| 32 | B | NT |
| 33 | B | A |
| 34 | A | A |
| 35 | A | A |
| 36 | B | A |
| 37 | B | A |
| 38 | B | A |
| 39 | B | A |
| 40 | A | A |
| 41 | A | A |
| 42 | A | A |
| 43 | A | A |
| 44 | A | A |
| 45 | A | A |
| 46 | A | A |
| 47 | NT | A |
| 48 | NT | A |
| 49 | B | A |
| 50 | B | NT |
| 51 | B | NR |
| 52 | B | NR |
| 53 | A | NR |
| 54 | B | NR |
| 55 | B | NR |
| 56 | A | NR |
| 57 | A | NR |
| 58 | A | NR |
| 59 | NT | NR |
| 60 | A | NR |
| 61 | A | NR |
| 62 | A | NR |
| 63 | A | NR |
| 64 | A | NR |
| 65 | A | NR |
| 66 | A | NR |
| 67 | A | NR |
| 68 | A | NR |
| 69 | A | NR |
| 70 | NT | NR |
| 71 | B | NR |

“NT” indicates the compound has not yet been tested in the assay in question.
“NR” indicates “Not Relevant”.
Examples 51-71 are the resultant carboxylic acid analogues of the amino acid esters that are cleaved inside cells. When these carboxylic acids are contacted with the cells, they do not penetrate into the cells and hence do not inhibit TNF-α in this assay.

TABLE 1
Macrophage selectivity benefit of Example 33

| | | | | Inhibition of |
|---|---|---|---|---|
| | | | | phosphorylation |
| | | Inhibition of | | of MAPKAP-2 in |
| | | P38α | | U937 cells (IC50, |
| Compound | Structure | (IC50 nM) | | nM) |
| Compound I | 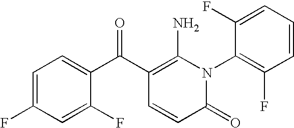 | 47 | | 9 |
| Example 33 | 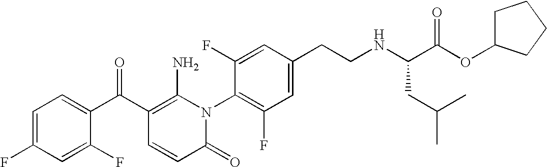 | Ester 100 | Acid 0.5 | 0.7 |

| | Inhibition of | Ratio |
|---|---|---|
| | phosphorylation | IC50s in |
| | of MAPKAP-2 in | HUT 78 |
| | HUT 78 cells | to U937 |
| Compound | (IC50, nM) | cells |
| Compound I | 10 | 1.1 |
| Example 33 | 73 | 112 |

In cells, p38 activity leads to the phosphorylation of the protein MAPKAP-2 and thus one method to assess the inhibition of p38 inside cells is to look at the decrease in the levels of phosphorylated MAPKAP-2. Table 1 lists the IC50s as measured by the level of MAPKAP-2 phosphorylation in a macrophage cell line (U937) and non-macrophage cell line (HUT78). For compound 1 (WO03076405) which lacks an esterase motif there is no difference between the IC50 in the macrophage and non-macrophage cell lines (9 nM vs 10 nM respectively). In contrast, example 33 that has an esterase motif that would be expected to confer macrophage selectivity, has an activity in the macrophage cell line (U937) which is 100 fold greater than in the non-macrophage cell line (HUT 78). It is therefore clear that example 33 exhibits a high degree of macrophage selectivity as compared to the compound lacking the esterase functionality.

1. A compound selected from the group consisting of:
Cyclopentyl (2S)-[(2-{4-[6-amino-5-(2,4-difluorobenzoyl)-2-oxopyridin-1(2H)-yl]phenyl}ethyl)amino](phenyl)acetate;
tert-butyl (2S)-{[2-(4-{6-amino-5-[(2,4-difluorophenyl)carbonyl]-2-oxopyridin-1(2H)-yl}phenyl)ethyl]amino}(phenyl)ethanoate;
Cyclopentyl (2S)-{[2-(4-{6-amino-5-[(2,4-difluorophenyl)carbonyl]-2-oxopyridin-1(2H)-yl}phenyl)ethyl]amino}(cyclohexyl)ethanoate;
tert-butyl (2S)-{[2-(4-{6-amino-5-[(2,4-difluorophenyl)carbonyl]-2-oxopyridin-1(2H)-yl}phenyl)ethyl]amino}(cyclohexyl)ethanoate;
Cyclopentyl N-[2-(4-{6-amino-5-[(2,4-difluorophenyl)carbonyl]-2-oxopyridin-1(2H)-yl}phenyl)ethyl]-L-valinate;
tert-butyl N-[2-(4-{6-amino-5-[(2,4-difluorophenyl)carbonyl]-2-oxopyridin-1(2H)-yl}phenyl)ethyl]-L-valinate;
Cyclopentyl N-[2-(4-{6-amino-5-[(2,4-difluorophenyl)carbonyl]-2-oxopyridin-1(2H)-yl}phenyl)ethyl]-3-methyl-L-valinate;
tert-butyl N-[2-(4-{6-amino-5-[(2,4-difluorophenyl)carbonyl]-2-oxopyridin-1(2H)-yl}phenyl)ethyl]-3-methyl-L-valinate;
Cyclopentyl N-[2-(4-{6-amino-5-[(2,4-difluorophenyl)carbonyl]-2-oxopyridin-1(2H)-yl}phenyl)ethyl]-D-leucinate;
tert-butyl N-[2-(4-{6-amino-5-[(2,4-difluorophenyl)carbonyl]-2-oxopyridin-1(2H)-yl}phenyl)ethyl]-D-leucinate;
Cyclopentyl N-[2-(4-{6-amino-5-[(2,4-difluorophenyl)carbonyl]-2-oxopyridin-1(2H)-yl}phenyl)ethyl]-O-tert-butyl-L-serinate;
tert-butyl N-[2-(4-{6-amino-5-[(2,4-difluorophenyl)carbonyl]-2-oxopyridin-1(2H)-yl}phenyl)ethyl]-O-tert-butyl-L-serinate;
(1R,2S,5S)-2-Isopropyl-5-methylcyclohexyl N-(2-{4-[6-amino-5-(2,4-difluorobenzoyl)-2-oxopyridin-1(2H)-yl]phenyl}ethyl)-L-leucinate;
(1S,2R,5S)-2-Isopropyl-5-methylcyclohexyl N-(2-{4-[6-amino-5-(2,4-difluorobenzoyl)-2-oxopyridin-1(2H)-yl]phenyl}ethyl)-L-leucinate;
Cyclopentyl N-[2-(4-{6-amino-5-[(2,4-difluorophenyl)carbonyl]-2-oxopyridin-1(2H)-yl}phenyl)ethyl]-O-tert-butyl-L-threoninate;
tert-butyl N-[2-(4-{6-amino-5-[(2,4-difluorophenyl)carbonyl]-2-oxopyridin-1(2H)-yl}phenyl)ethyl]-O-tert-butyl-L-threoninate;
Cyclopentyl N-[2-(4-{6-amino-5-[(2,4-difluorophenyl)carbonyl]-2-oxopyridin-1(2H)-yl}phenyl)ethyl]-L-threoninate;
Cyclopentyl N-[2-(4-{6-amino-5-[(2,4-difluorophenyl)carbonyl]-2-oxopyridin-1(2H)-yl}phenyl)ethyl]-L-isoleucinate;
tert-butyl N-[2-(4-{6-amino-5-[(2,4-difluorophenyl)carbonyl]-2-oxopyridin-1(2H)-yl}phenyl)ethyl]-L-isoleucinate;
Cyclopentyl N-[2-(4-{6-amino-5-[(2,4-difluorophenyl)carbonyl]-2-oxopyridin-1(2H)-yl}phenyl)ethyl]-L-alaninate;
tert-butyl N-[2-(4-{6-amino-5-[(2,4-difluorophenyl)carbonyl]-2-oxopyridin-1(2H) yl}phenyl)ethyl]-L-alaninate;
Cyclopentyl N-[2-(4-{6-amino-5-[(2,4-difluorophenyl)carbonyl]-2-oxopyridin-1(2H)-yl}phenyl)ethyl]-L-phenylalaninate;
tert-butyl N-[2-(4-{6-amino-5-[(2,4-difluorophenyl)carbonyl]-2-oxopyridin-1(2H)-yl}phenyl)ethyl]-L-phenylalaninate;
Cyclopentyl N-(2-{4-[6-amino-5-(4-fluorobenzoyl)-2-oxopyridin-1(2H)-yl]-3,5-difluorophenyl}ethyl)-L-leucinate;
tert-butyl N-(2-{4-[6-amino-5-(4-fluorobenzoyl)-2-oxopyridin-1(2H)-yl]-3,5-difluorophenyl}ethyl)-L-leucinate;
Cyclopentyl (2S)-[(2-{4-[6-amino-5-(4-fluorobenzoyl)-2-oxopyridin-1(2H)-yl]-3,5-difluorophenyl}ethyl)amino](phenyl)acetate;
tert-butyl (2S)-[(2-{4-[6-amino-5-(4-fluorobenzoyl)-2-oxopyridin-1(2H)-yl]-3,5-difluorophenyl}ethyl)amino](phenyl)acetate;
Cyclopentyl (2S)-[(2-{4-[6-amino-5-(4-fluorobenzoyl)-2-oxopyridin-1(2H)-yl]-3,5-difluorophenyl}ethyl)amino](cyclohexyl)acetate;
Cyclopentyl N-(2-{4-[6-amino-5-(4-fluorobenzoyl)-2-oxopyridin-1(2H)-yl]-3,5-difluorophenyl}ethyl)-D-leucinate;
tert-butyl N-(2-{4-[6-amino-5-(4-fluorobenzoyl)-2-oxopyridin-1(2H)-yl]-3,5-difluorophenyl}ethyl)-D-leucinate;
Cyclopentyl N-(2-{4-[6-amino-5-(4-fluorobenzoyl)-2-oxopyridin-1(2H)-yl]-3,5-difluorophenyl}ethyl)-O-tert-butyl-L-serinate;
tert-butyl N-(2-{4-[6-amino-5-(4-fluorobenzoyl)-2-oxopyridin-1(2H)-yl]-3,5-difluorophenyl}ethyl)-O-tert-butyl-L-serinate;
cyclopentyl N-(2-{4-[6-amino-5-(2,4-difluorobenzoyl)-2-oxopyridin-1(2H)-yl]-3,5-difluorophenyl}ethyl)-L-leucinate;
Cyclopentyl (2S)-[(2-{4-[6-amino-5-(2,4-difluorobenzoyl)-2-oxopyridin-1(2H)-yl]-3,5-difluorophenyl}ethyl)amino](phenyl)acetate;
tert-butyl (2S)-[(2-{4-[6-amino-5-(2,4-difluorobenzoyl)-2-oxopyridin-1(2H)-yl]-3,5-difluorophenyl}ethyl)amino](phenyl)acetate;
Cyclopentyl (2S)-[(2-{4-[6-amino-5-(2,4-difluorobenzoyl)-2-oxopyridin-1(2H)-yl]-3,5-difluorophenyl}ethyl)amino](cyclohexyl)acetate;
tert-butyl N-(2-{4-[6-amino-5-(2,4-difluorobenzoyl)-2-oxopyridin-1(2H)-yl]-3,5-difluorophenyl}ethyl)-L-leucinate;
Cyclopentyl N-(2-{4-[6-amino-5-(2,4-difluorobenzoyl)-2-oxopyridin-1(2H)-yl]-3,5-difluorophenyl}ethyl)-D-leucinate;
tert-butyl N-(2-{4-[6-amino-5-(2,4-difluorobenzoyl)-2-oxopyridin-1(2H)-yl]-3,5-difluorophenyl}ethyl)-D-leucinate;
Cyclopentyl N-(2-{4-[6-amino-5-(2,4-difluorobenzoyl)-2-oxopyridin-1(2H)-yl]-3,5-difluorophenyl}ethyl)-O-tert-butyl-L-serinate
tert-butyl N-(2-{4-[6-amino-5-(2,4-difluorobenzoyl)-2-oxopyridin-1(2H)-yl]-3,5-difluorophenyl}ethyl)-O-tert-butyl-L-serinate;
Cyclopentyl (2R)-[(2-{4-[6-amino-5-(2,4-difluorobenzoyl)-2-oxopyridin-1(2H)-yl]-3,5-difluorophenyl}ethyl)amino](phenyl)acetate;
Cyclopentyl N-[2-(4-{6-amino-5-[(2,4-difluorophenyl)carbonyl]-2-oxopyridin-1(2H)-yl}-3,5difluorophenyl)ethyl]-L-valinate;
Cyclopentyl (2S)-{[2-(4-{6-amino-5-[(2,4-difluorophenyl)carbonyl]-2-oxopyridin-1(2H)-yl}-3,5-difluorophenyl)ethyl]amino}(4-hydroxyphenyl)ethanoate;
Cyclopentyl N-[2-(4-{6-amino-5-[(2,4-difluorophenyl)carbonyl]-2-oxopyridin-1(2H)-yl}-3,5-difluorophenyl)ethyl]-L-threoninate;
Cyclopentyl (25)-{[2-(4-{6-amino-5-[(2,4-difluorophenyl)carbonyl]-2-oxopyridin-1(2H)-yl}-3,5-difluorophenyl)ethyl]amino}(4-methoxyphenyl)ethanoate;
Cyclopentyl (2S)-{[2-(4-{6-amino-5-[(2,4-difluorophenyl)carbonyl]-2-oxopyridin-1(2H)-yl}-3,5-difluorophenyl)ethyl]amino}(4-fluorophenyl)ethanoate;
tert-butyl (2S)-{[2-(4-{6-amino-5-[(2,4-difluorophenyl)carbonyl]-2-oxopyridin-1(2H)-yl}-3,5-difluorophenyl)ethyl]amino}(4-fluorophenyl)ethanoate;
Cyclopentyl N-(2-{5-[6-amino-5-(2,4-difluorobenzoyl)-2-oxopyridin-1(2H)-yl]-2-thienyl}ethyl)-L-leucinate;
tert-butyl N-(2-{5-[6-amino-5-(2,4-difluorobenzoyl)-2-oxopyridin-1(2H)-yl]-2-thienyl}ethyl)-L-leucinate;
(2S)-[(2-{4-[6-amino-5-(2,4-difluorobenzoyl)-2-oxopyridin-1(2H)yl]-phenyl}ethyl)amino](phenyl)acetic acid;
(2S)-{[2-(4-{6-amino-5-[(2,4-difluorophenyl)carbonyl]-2-oxopyridin-1(2H)-yl}phenyl)ethyl]amino}(cyclohexyl)ethanoic acid;
N-[2-(4-{6-amino-5-[(2,4-difluorophenyl)carbonyl]-2-oxopyridin-1(2H)-yl}phenyl)ethyl]-L-valine;
N-[2-(4-{6-amino-5-[(2,4-difluorophenyl)carbonyl]-2-oxopyridin-1(2H)-yl}phenyl)ethyl]-3-methyl-L-valine;
N-[2-(4-{6-amino-5-[(2,4-difluorophenyl)carbonyl]-2-oxopyridin-1(2H)-yl}phenyl)ethyl]-D-leucine;
N-[2-(4-{6-amino-5-[(2,4-difluorophenyl)carbonyl]-2-oxopyridin-1(2H)-yl}phenyl)ethyl]-O-tert-butyl-L-serine;
N-[2-(4-{6-amino-5-[(2,4-difluorophenyl)carbonyl]-2-oxopyridin-1(2H)-yl}phenyl)ethyl]-L-serine;
N-[2-(4-{6-amino-5-[(2,4-difluorophenyl)carbonyl]-2-oxopyridin-1(2H)-yl}phenyl)ethyl]-L-threonine;
N-[2-(4-{6-amino-5-[(2,4-difluorophenyl)carbonyl]-2-oxopyridin-1(2H)-yl}phenyl)ethyl]-L-isoleucine;
N-[2-(4-{6-amino-5-[(2,4-difluorophenyl)carbonyl]-2-oxopyridin-1(2H)-yl}phenyl)ethyl]-L-alanine;
N-[2-(4-{6-amino-5-[(2,4-difluorophenyl)carbonyl]-2-oxopyridin-1(2H)-yl}phenyl)ethyl]-L-phenylalanine;
(2S)-[(2-{4-[6-amino-5-(4-fluorobenzoyl)-2-oxopyridin-1(2H)-yl]-3,5-difluorophenyl}ethyl)amino](phenyl)acetic acid;
N-(2-{4-[6-amino-5-(4-fluorobenzoyl)-2-oxopyridin-1(2H)-yl]-3,5-difluorophenyl}ethyl)-L-leucine;
N-(2-{4-[6-amino-5-(4-fluorobenzoyl)-2-oxopyridin-1(2H)-yl]-3,5-difluorophenyl}ethyl)-D-leucine;
N-(2-{4-[6-amino-5-(4-fluorobenzoyl)-2-oxopyridin-1(2H)-yl]-3,5-difluorophenyl}ethyl)-O-tert-butyl-L-serine;
(2S)-[(2-{4-[6-amino-5-(2,4-difluorobenzoyl)-2-oxopyridin-1(2H)-yl]-3,5-difluorophenyl}ethyl)amino](phenyl)acetic acid;
N-(2-{4-[6-amino-5-(2,4-difluorobenzoyl)-2-oxopyridin-1(2H)-yl]-3,5-difluorophenyl}ethyl)-L-leucine;
N-(2-{4-[6-amino-5-(2,4-difluorobenzoyl)-2-oxopyridin-1(2H)-yl]-3,5-difluorophenyl}ethyl)-D-leucine;
N-(2-{4-[6-amino-5-(2,4-difluorobenzoyl)-2-oxopyridin-1(2H)-yl]-3,5-difluorophenyl}ethyl)-O-tert-butyl-L-serine;
(2S)-{[2-(4-[6-amino-5-[(2,4-difluorophenyl)carbonyl]-2-oxopyridin-1(2H)-yl]-3,5-difluorophenyl)ethyl]amino}(4-fluorophenyl)ethanoic acid and
N-(2-{5-[6-amino-5-(2,4-difluorobenzoyl)-2-oxopyridin-1(2H)-yl]-2-thienyl}ethyl)-L-leucine.

2. A compound as claimed in claim 1 which is in the form of a pharmaceutically acceptable salt.

3. A pharmaceutical composition comprising a compound as claimed in claim 1, together with a pharmaceutically acceptable carrier.

4. The composition of claim 3 wherein the compound is present in an amount effective for inhibiting the activity of a p38 MAP kinase enzyme in vitro or in vivo.

5. The composition of claim 3 wherein the compound is present in an amount effective for the treatment of autoimmune or inflammatory disease

6. A method of inhibiting the activity of a p38 MAP kinase enzyme comprising contacting the enzyme with an amount of a compound as claimed in claim 1 effective for such inhibition.

7. A method for the treatment of autoimmune or inflammatory disease which comprises administering to a subject suffering such disease an effective amount of a compound as claimed in claim 1.

8. The method as claimed in claim 7 wherein the disease is psoriasis, inflammatory bowel disease, Crohn's disease, ulcerative colitis, chronic obstructive pulmonary disease, asthma, multiple sclerosis, diabetes, atopic dermatitis, graft versus host disease, or systemic lupus erythematosus.

9. The method as claimed in claim 7 wherein the disease is rheumatoid arthritis.

10. A pharmaceutical composition comprising a compound as claimed in claim 2, together with a pharmaceutically acceptable carrier.

11. A method of inhibiting the activity of a p38 MAP kinase enzyme comprising contacting the enzyme with an amount of a compound as claimed in claim 2 effective for such inhibition.

12. A method for the treatment of autoimmune or inflammatory disease which comprises administering to a subject suffering such disease an effective amount of a compound as claimed in claim 2.

13. The method as claimed in claim 12 wherein the disease is psoriasis, inflammatory bowel disease, Crohn's disease, ulcerative colitis, chronic obstructive pulmonary disease, asthma, multiple sclerosis, diabetes, atopic dermatitis, graft versus host disease, or systemic lupus erythematosus.

14. The method as claimed in claim 12 wherein the disease is rheumatoid arthritis.
